SEQUENCE LISTING (25 sequences)

SEQ ID NO: 1 - protein, 5 aa, Artificial Sequence
  conserved C-terminal pentapeptide motif
  MISC_FEATURE (2)..(2): Xaa = His, Asn, Ser or Tyr
  MISC_FEATURE (3)..(3): Xaa = Ser, Pro or Ala
FXXWG

SEQ ID NO: 2 - protein, 6 aa, Artificial Sequence
  C-terminal motif
WFGPRL

SEQ ID NO: 3 - protein, 5 aa, Artificial Sequence
  C-terminal motif
  misc_feature (2)..(2): Xaa can be any naturally occurring amino acid
FXPRL

SEQ ID NO: 4 - protein, 7 aa, Artificial Sequence
  peptide
  MISC_FEATURE (2)..(2): Xaa = G or T
  MISC_FEATURE (6)..(6): Xaa = I or V
AXAPRXB

SEQ ID NO: 5 - protein, 6 aa, Artificial Sequence
  peptide
  MISC_FEATURE (2)..(2): Xaa = G or T
FXCPRL

SEQ ID NO: 6 - protein, 5 aa, Artificial Sequence
  peptide
FTPRI

SEQ ID NO: 7 - protein, 5 aa, Artificial Sequence
  peptide
FKPRL

SEQ ID NO: 8 - protein, 5 aa, Artificial Sequence
  peptide
FTPRV

SEQ ID NO: 9 - protein, 4 aa, Artificial Sequence
  peptide
  MISC_FEATURE (2)..(2): Xaa = T with hydroxyproline (Hyp) side chain
FXRV

SEQ ID NO: 10 - protein, 4 aa, Artificial Sequence
  peptide
  MISC_FEATURE (2)..(2): Xaa = T with an Octahydroindole-2-carboxylic acid (Oic) side chain
FXRV

SEQ ID NO: 11 - protein, 5 aa, Artificial Sequence
  peptide
  MISC_FEATURE (5)..(5): Xaa = I or V
ATPRX

SEQ ID NO: 12 - protein, 5 aa, Artificial Sequence
  peptide
ATPRI

SEQ ID NO: 13 - protein, 5 aa, Artificial Sequence
  peptide
FGPRL

SEQ ID NO: 14 - protein, 10 aa, Artificial Sequence
  peptide
  MISC_FEATURE (4)..(4): Xaa = L, beta-homoleucine, beta-homoalanine or beta-homo-phenylalanine
  MISC_FEATURE (5)..(5): Xaa = V, beta-homoleucine, beta-homvaline, beta-homoalanine or beta-homo-phenylalanine
  MISC_FEATURE (6)..(6): Xaa = A, beta-alanine
ASGXXXFPRV

SEQ ID NO: 15 - protein, 10 aa, Artificial Sequence
  peptide
  MISC_FEATURE (4)..(4): Xaa = L, beta-homoleucine, beta-homo-alanine or beta-homo-phenylalanine
ASGXVAFPRV

SEQ ID NO: 16 - protein, 10 aa, Artificial Sequence
  peptide
  MISC_FEATURE (5)..(5): Xaa = V, beta-homoleucine, beta-homovaline, beta-homoalanine or beta-homophenylalanine
ASGLXAFPRV

SEQ ID NO: 17 - protein, 10 aa, Artificial Sequence
  peptide
  MISC_FEATURE (4)..(4): Xaa = L or beta-homoleucine, beta-homo-alanine or beta-homo-phenylalanine
  MISC_FEATURE (6)..(6): Xaa = A or beta-alanine
ASGXVXFPRV

SEQ ID NO: 18 - protein, 10 aa, Artificial Sequence
  peptide
  MISC_FEATURE (4)..(4): Xaa = beta-homoleucine
ASGXVAFPRV

SEQ ID NO: 19 - protein, 10 aa, Artificial Sequence
  peptide
  MISC_FEATURE (5)..(5): Xaa = beta-homoleucine
ASGLXAFPRV

SEQ ID NO: 20 - protein, 10 aa, Artificial Sequence
  peptide
  MISC_FEATURE (4)..(4): Xaa = beta-homoleucine
  MISC_FEATURE (6)..(6): Xaa = beta-alanine
ASGXVXFPRV

SEQ ID NO: 21 - protein, 16 aa, Artificial Sequence
  kinin
CNSVVLGKKQRFHSWG

SEQ ID NO: 22 - protein, 12 aa, Artificial Sequence
  CAPA-1
GANMGLYAFPRV

SEQ ID NO: 23 - protein, 13 aa, Artificial Sequence
  labelled CAPA-1
CGANMGLYAFPRV

SEQ ID NO: 24 - protein, 4 aa, Artificial Sequence
  kinin 1728
  MISC_FEATURE (1)..(1): Xaa = F with alpha-aminoisobutyric acid (Aib) side chain
  misc_feature (2)..(2): Xaa can be any naturally occurring amino acid
  MISC_FEATURE (4)..(4): Xaa = F with alpha-aminoisobutyric acid (Aib) side chain
XXWG

SEQ ID NO: 25 - protein, 4 aa, Artificial Sequence
  Kinin 2139
  MISC_FEATURE (2)..(2): Xaa = F with alpha-aminoisobutyric acid (Aib) side chain
FXWG

(19) United States
(12) Patent Application Publication
(10) Pub. No.: US 2022/0039395 A1
(43) Pub. Date: Feb. 10, 2022

(54) INSECT CONTROL AGENTS

(71) Applicants: THE UNIVERSITY COURT OF THE UNIVERSITY OF GLASGOW, Glasgow Strathclyde (GB); THE USA, AS REPRESENTED BY THE SEC OF AGRICULTURE, Washington D.C., WA (US)

(72) Inventors: Lucy ALFORD, Glasgow Strathclyde (GB); Julian A. DOW, Glasgow Strathclyde (GB); Shireen A. DAVIES, Glasgow Strathclyde (GB); Ronald J. NACHMAN, College Station, TX (US)

(21) Appl. No.: 17/299,201

(22) PCT Filed: Dec. 3, 2019
(86) PCT No.: PCT/EP2019/083553

§ 371 Date: Jun. 2, 2021

Related U.S. Application Data
(60) Provisional application No. 62/774,546, filed on Dec. 3, 2018.

(51) Int. Cl. A01N 63/50; A01N 37/46; C07K 14/435; A01P 7/04

(52) U.S. Cl. CPC A01N 63/50; A01P 7/04; C07K 14/43563; A01N 37/46

(57) ABSTRACT

The invention relates to CAP2b analogues having activity against hemipteran insects such as aphids, and their use as insect control agents (e.g. insecticides) and plant protection agents. In particular it has been found that a known CAP2b analogue designated 1895, and new CAP2b analogues including molecules designated 2129, 2315, 2316 and 2320, have activity against hemipteran insects and so find use for control of hemipteran insects and plant protection.

### CROSS-REFERENCE

This application is a 371 National Stage filing and claims benefit under 35 U.S.C. § 120 to International Application No. PCT/EP2019/083553 filed Dec. 3, 2019, which claims priority to U.S. Provisional Application No. 62/774,546, filed Dec. 3, 2018, each of which is incorporated herein by reference in its entirety.

### FIELD OF THE INVENTION

The present invention relates to CAP2b analogues having activity against hemipteran insects such as aphids, and their use as insect control agents (e.g. insecticides) and plant protection agents.

### BACKGROUND

With a global dependence on broad-spectrum insecticides, the damaging effects of which are well documented,^{1,2} there is increasing need for the development of greener, target-specific insecticides. The development and employment of neuropeptide synthetic analogues offers a promising avenue in the drive for greener and target-specific insecticidal agents. Within the insects, neuropeptides are regulatory peptides with functional roles in growth and development, behaviour and reproduction, metabolism and homeostasis, and muscle movement.³ Due to their high specificity, neuropeptides and their cognate receptors (G-protein coupled receptors, GPCRs) may be developed towards insecticidal agents^{4,6} to selectively reduce the fitness of target pest insects, whilst minimising detrimental environmental impacts.

Insect neuropeptide families include the insect kinins and cardio acceleratory peptides (CAPA, CAP2b) neuropeptides.

Insect kinins are multifunctional neuropeptides which share a conserved C-terminal pentapeptide motif Phe-X¹-X²-Trp-Gly-NH2 (SEQ ID NO: 1), where X¹ can be His, Asn, Ser or Tyr, and X² can be Ser, Pro or Ala.⁶ The insect kinins have been identified in most insects, with the exception of Coleoptera,⁷ and have diverse roles in the stimulation of muscle,⁸ fluid secretion in renal tubules,^{9,10} digestive enzyme release,¹¹ inhibition of larval weight gain¹² and the desiccation and starvation stress response.^{13,14}

The second family, the CAPA peptides, were first identified from the moth Manduca sexta (CAP2b)¹⁵ and have since been identified in many insect families.¹⁶ Although function varies depending on insect species, life stage, and lifestyle, CAPA peptides play a key role in myomodulation and osmoregulation¹⁶ and have more recently been linked to desiccation and cold tolerance in Drosophila species.^{17,18}

The CAPA peptides belong to the PRXamide superfamily which can be further subdivided into three major classes: CAPA peptides, pyrokinins (PK) and ecdysis triggering hormone (ETH).¹⁹ The pyrokinins are further subdivided into diapause hormone (DH) and pheromone biosynthesis activating neuropeptides (PBAN) and by their C-terminal motifs WFGPRLamide (SEQ ID NO: 2) and FXPRLamide (SEQ ID NO: 3) respectively.²⁰ The GPCRs of this ligand group form a homologous cluster, suggesting co-evolution of ancestrally related ligand-receptor partners. As a result, some cross activity by analogues of the ligand sub-groups with respective, recombinant receptors has been observed.^{21,22}

### SUMMARY OF THE INVENTION

The inventors have discovered that a known CAP2b analogue designated 1895, and new CAP2b analogues including those designated 2129 and 2125, have activity against hemipteran insects and so find use as insect control agents (e.g. insecticides), particularly for targeting hemipteran insects, and plant protection agents.

Thus, in a first aspect, the invention provides the use, as an insect control agent against hemipteran insects, of a compound having the formula:

R¹-L¹-Z^{a}—Z—R²

wherein:
Z^{a} is a peptide of 1 to 8 amino acids, or is absent;
Z is a peptide having a sequence selected from:

(SEQ ID NO: 4)
A-Xa-PR-Xb;
(SEQ ID NO: 5)
F-Xc-PRL;

where Xa and Xc are independently G or T and Xb is I or V;

(SEQ ID NO: 6)
FTPRI;
(SEQ ID NO: 7)
FKPRL;
(SEQ ID NO: 8)
FTPRV;
(SEQ ID NO: 9)
FT[Hyp]RV;
and
(SEQ ID NO: 10)
FT[Oic]RV;

L¹ is absent or is selected from C₁₋₆-alkylene, C₁₋₆-alkenylene and (poly)alkyleneglycol where each L¹ if present may be optionally substituted with one or more groups selected from oxo (═O), halogen, ═N and ═S;

R¹ is hydrogen (which may be designated “H—” or “Hy-”), C₁₋₄ alkyl (e.g. methyl, ethyl, propyl, butyl), acetyl, formyl, benzoyl or trifluoroacetyl, —NHC₁₋₁₈-alkyl, —NHC₆₋₁₆-Aryl, or —NH—C₁₋₆-alkyl-C₆₋₁₀aryl, each of which may optionally be substituted with one or more groups selected from halogen, C₁₋₆-alkyl, or C₁₋₆-haloalkyl;

and R² is NH₂ or OR²a wherein Rea is C₁₋₆-alkyl (e.g. methyl, ethyl, propyl, butyl, pentyl or hexyl), Cm-alkenyl, C₆₋₁₆-aryl, C₆₋₁₆-aryl-C₁₋₆-alkyl, C₁₋₆-alkyl-C₆₋₁₆-aryl, or C₁₋₆-haloalkyl, each of which may optionally be substituted with one or more groups selected from halogen, C₁₋₆-alkyl, or C₁₋₆-haloalkyl.

In some embodiments, Z may have the formula ATPR-Xb (SEQ ID NO: 11), where Xb is I or V. For example, Z may have the formula ATPRI (SEQ ID NO: 12).

In other embodiments, Z is FGPRL (SEQ ID NO: 13).

Za is an optional additional peptide sequence of 1 to 8 amino acids in length. Thus it may be 1, 2, 3, 4, 5, 6, 7, or 8 residues in length. In some embodiments, it may be desirable that Z^{a} is composed primarily or entirely of small residues such as Ser, Gly and Ala, e.g. at least half of the residues in Z^{a} may be selected from Ser, Gly and Ala.

In some embodiments, Z^{a} is absent.

In some embodiments, L¹ is C₁₋₆-alkylene optionally substituted with one or more groups selected from oxo (═O), halogen, ═N and ═S. For example, L¹ may be substituted with one or more oxo group.

In some embodiments L¹ is —(C═O)C₁₋₄-alkylene-(C═O)—, e.g. L¹ is

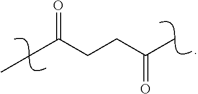

In some embodiments R¹ is hydrogen, —NHC₁₋₁₈-alkyl, —NHC₆₋₁₆-Aryl, or —NH—C₁₋₆-alkyl-C₆₋₁₆aryl optionally substituted with one or more groups selected from halogen, C₁₋₆-alkyl, or C₁₋₆-haloalkyl. For example R¹ may be —NHC₆₋₁₆-Aryl optionally substituted with one or more groups selected from halogen, C₁₋₆-alkyl, or C₁₋₆-haloalkyl.

For example, R¹ may be selected from N(H)npropyl, —N(H)ipropyl, —N(H)nbutyl, —N(H)isoamyl, —N(H)nhexyl, —N(H)noctyl, —N(H)t-octyl, —N(H)ndecyl, —N(H)ndodecyl, or N(H)-fluorenyl optionally substituted with one or more halogen groups. For example, R¹ may be —N(H)-fluorenyl substituted with one or more bromine atom, e.g.:

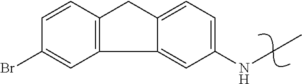 [SMILES: CCNc1ccc2c(c1)-c1cc(Br)ccc1C2]

For example, L¹-R¹ may be:

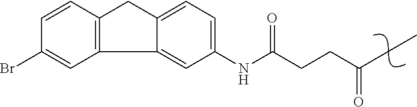 [SMILES: CCC(=O)CCC(=O)Nc1ccc2c(c1)-c1cc(Br)ccc1C2]

which is referred to elsewhere in this specification by the notation “2Abf-Suc”.

In some embodiments, R² is NH₂

Thus, the compound may be:

(SEQ ID NO: 12)
2Abf-Suc-ATPRI-NH₂;
(SEQ ID NO: 13)
2Abf-Suc-FGPRL-NH₂;
(SEQ ID NO: 6)
2Abf-Suc-FTPRI-NH₂;
(SEQ ID NO: 7)
2Abf-Suc-FKPRL-NH₂;
(SEQ ID NO: 8)
2Abf-Suc-FTPRV-NH₂,
(SEQ ID NO: 9)
2Abf-Suc-FT[Hyp]RV-NH₂;
or
(SEQ ID NO: 10)
2Abf-Suc-FT[Oic]RV-NH₂.

The compounds have activity against hemipteran insects.

The compounds typically increase insect mortality, in general, or under conditions of stress such as cold stress, desiccation stress or starvation stress. When used to increase insect mortality, the compounds described (and compositions containing them) may be regarded as insecticides. The compounds may also have activity in reducing insect fecundity, whether of individual insects or of an insect population as a whole. The effect on fecundity may be exerted in conjunction with an effect on mortality or independently thereof.

Without wishing to be bound by theory, any or all of the effects described may be mediated by binding of the compounds to the CAP2b receptor of the target hemipteran insects. The CAP2b receptor of M. persicae may be used as a model system, as described in the examples below. Thus, it may be preferable that the compounds have affinity for the CAP2b receptor, e.g. for the CAP2b receptor of M. persicae. In some embodiments, they have an agonistic effect on the CAP2b signalling pathway. In other embodiments, they may have an antagonistic effect on the CAP2b signalling pathway. The term “CAPA” is now in more common use than the term “CAP2b”. The terms “CAP2b” and “CAPA” may be used interchangeably, as may “CAP2b receptor” and “CAPA receptor”.

The invention provides a method of increasing hemipteran insect mortality comprising contacting a hemipteran insect or hemipteran insect population with a compound as described.

The invention further provides a method of reducing cold tolerance, reducing desiccation stress tolerance, reducing starvation stress tolerance, and/or reducing fecundity of a hemipteran insect, or of a hemipteran insect population, comprising contacting a hemipteran insect or insect population with a compound as described.

The insect or insect population may be undergoing conditions of cold, desiccation stress, or starvation stress, as appropriate.

The compound may be applied directly to an insect or insect population. For example, it may be applied topically. Alternatively, the compound may be applied indirectly. For example, it may be applied to a substrate likely to come into contact with an insect or insect population. The substrate may be a plant, especially for Hemiptera which represent pests of plants (whether crops or horticultural plants). However, for Hemiptera which represent pests to humans, such as the Cimicidae family (e.g. bedbugs of the genus Cimex, such as Cimex lectularius) or the Reduviidae family (e.g. of the genus Rhodnius such as Rhodnius prolixus, or Triatoma such as Triatoma infestans) which can be vectors of human disease, the substrate may be a domestic surface or article, such as bedding, a mattress, or any other suitable domestic surface. The compound may be applied to the substrate in a form suitable for ingestion by an insect.

The invention further provides the use of a compound as described as a plant protection agent, and specifically for protecting a plant against hemipteran insects.

The invention further provides a method of inhibiting infestation of a plant by hemipteran insects comprising contacting the plant with a compound as described.

The method may be prophylactic. Thus, for example, the compound may be applied to the plant while the plant is free or substantially free of hemipteran insects.

Alternatively, the plant may already be colonised or infested by hemipteran insects. Thus, the invention further provides a method of reducing infestation of a plant, or of reducing hemipteran insect load on a plant, the method comprising contacting the plant with a compound as described.

In any of these embodiments, the compound may be provided as part of a composition, such as an insect control composition (e.g. insecticide composition) or a plant protection composition. Reference to application or use of a compound should therefore be construed as encompassing application or use of a suitable composition, unless the context demands otherwise.

The composition typically comprises a compound as described in combination with one or more ancillary component such as solvents, carriers, diluents, adjuvants, preservatives, dispersants, emulsifying agents, or synergists.

The composition may further comprise one or more additional active insecticides.

In a second aspect, the invention further provides a compound having the formula:

R¹-L¹-Z^{a}—Z—R²

wherein:
Z^{a} is a peptide of 1 to 12 amino acids, or is absent;
Z is a peptide having a sequence selected from:
A-Xa-PR-Xb (SEQ ID NO: 4), where Xa is G or T and Xb is I or V;

(SEQ ID NO: 8)
FTPRV;
(SEQ ID NO: 9)
FT[Hyp]RV;
and
(SEQ ID NO: 10)
FT[Oic]RV.

L¹ is absent or is selected from C₁₋₆-alkylene, C₁₋₆-alkenylene and (poly)alkyleneglycol where each L¹ if present may be optionally substituted with one or more groups selected from oxo (═O), halogen, ═N and ═S.

R¹ is hydrogen, C₁₋₄ alkyl (e.g. methyl, ethyl, propyl, butyl), acetyl, formyl, benzoyl or trifluoroacetyl, —NHC₁₋₁₈-alkyl, —NHC₆₋₁₆-Aryl, or —NH—C₁₋₆-alkyl-C₆₋₁₀aryl, each of which may optionally be substituted with one or more groups selected from halogen, C₁₋₆-alkyl, or C₁₋₆-haloalkyl.

and R² is NH₂ or OR^{2a} wherein Rea is C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkyl-C₆₋₁₆-aryl, or C₁₋₆-haloalkyl, each of which may optionally be substituted with one or more groups selected from halogen, C₁₋₆-alkyl, or C₁₋₆-haloalkyl.

The peptide Z may have the formula ATPR-Xb (SEQ ID NO: 11), where Xb is I or V.

The peptide Z may have the formula ATPRI (SEQ ID NO: 12).

In some embodiments, Z^{a} is absent.

In some embodiments, L¹ is C₁₋₆-alkylene optionally substituted with one or more groups selected from oxo (═O), halogen, ═N and ═S. For example, L¹ may be substituted with one or more oxo group.

In some embodiments L¹ is —(C═O)C₁₋₄-alkylene-(C═O)—, e.g. L¹ is

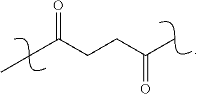

In some embodiments R¹ is —NHC₁₋₁₈-alkyl, —NHC₆₋₁₆-Aryl, or —NH—C₁₋₆-alkyl-C₆₋₁₆aryl optionally substituted with one or more groups selected from halogen, C₁₋₆-alkyl, or C₁₋₆-haloalkyl. For example R¹ may be —NHC₆₋₁₆-Aryl optionally substituted with one or more groups selected from halogen, C₁₋₆-alkyl, or C₁₋₆-haloalkyl.

For example, R¹ may be selected from N(H)npropyl, —N(H)ipropyl, —N(H)nbutyl, —N(H)isoamyl, —N(H)nhexyl, —N(H)noctyl, —N(H)t-octyl, —N(H)ndecyl, —N(H)ndodecyl, or N(H)-fluorenyl optionally substituted with one or more halogen groups. For example, R¹ may be —N(H)-fluorenyl substituted with one or more bromine atom, e.g.:

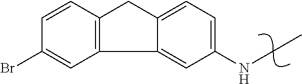 [SMILES: CCNc1ccc2c(c1)-c1cc(Br)ccc1C2]

For example, L¹-R¹ may be:

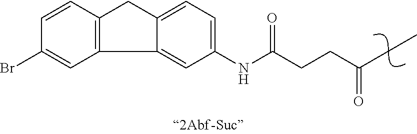 [SMILES: CCC(=O)CCC(=O)Nc1ccc2c(c1)-c1cc(Br)ccc1C2]

In some embodiments, R² is NH₂

The compound may be:

(SEQ ID NO: 12)
2Abf-Suc-ATPRI-NH₂;
(SEQ ID NO: 8)
2Abf-Suc-FTPRV-NH₂;
(SEQ ID NO: 9)
2Abf-Suc-FT[Hyp]RV-NH₂;
or
(SEQ ID NO: 10)
2Abf-Suc-FT[Oic]RV-NH₂.

The invention further provides a composition, e.g. an insect control composition or plant protection composition, comprising a compound of the second aspect of the invention in admixture with one or more solvents, carriers, diluents, adjuvants, preservatives, dispersants, emulsifying agents, or synergists. The composition may be an aqueous composition.

The inventors have also discovered further CAPA/Cap2b peptides capable of acting as insect control agents, particularly against hemipteran insects. These include the peptides designated 2315, 2316 and 2320.

In a third aspect, the invention further provides a compound having the formula:

R¹-L¹-Z^{a}—Z—R²

wherein:

Zₐ is a peptide of 1 to 12 amino acids, or is absent;

Z is a peptide having the formula:

(SEQ ID NO: 14)
ASG-X4-X5-X6-FPRV

wherein:
X4 is L, [βhL], [βhA] or [βhF];
X5 is V, [βhL], [βhV], [βhA] or [βhF];

### X6 is A or [βA].

L¹ is absent or is selected from C₁₋₆-alkylene, C₁₋₆-alkenylene and (poly)alkyleneglycol where each L¹ if present may be optionally substituted with one or more groups selected from oxo (═O), halogen, ═N and ═S.

R¹ is hydrogen (which may be designated “H” or “Hy”), C₁₋₄ alkyl (e.g. methyl, ethyl, propyl, butyl), acetyl, formyl, benzoyl or trifluoroacetyl, —NHC₁₋₁₈-alkyl, —NHC₆₋₁₆-Aryl, or —NH—C₁₋₆-alkyl-C₆₋₁₀aryl, each of which may optionally be substituted with one or more groups selected from halogen, C₁₋₆-alkyl, or C₁₋₆-haloalkyl.

and R² is NH₂ or OR²a wherein Rea is C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkyl-C₆₋₁₆-aryl, or C₁₋₆-haloalkyl, each of which may optionally be substituted with one or more groups selected from halogen, C₁₋₆-alkyl, or C₁₋₆-haloalkyl.

The peptide Z may have the formula:

ASG-X4-VAFPRV (SEQ ID NO: 15), wherein X4 is L, [βhL], [βhA] or [βhF];
ASGL-X5-AFPRV (SEQ ID NO: 16), wherein X5 is V, [βhL], [βhV], [βhA] or [βhF]; or
ASG-X4-V-X6-FPRV (SEQ ID NO: 17) wherein X4 is L, [βhL], [βhA] or or [βhF]; and

### X6 is A or [βA].

In some embodiments

X4 is L or [βhL];
X5 is V or [βhL];

### X6 is A or [βA].

The peptide Z may have the sequence:

(SEQ ID NO: 18)
ASG[βhL]VAFPRV;
(SEQ ID NO: 19)
ASGL[βhL]AFPRV;
or
(SEQ ID NO: 20)
ASG[βhL]V[βA]FPRV.

In some embodiments, Z^{a} is absent.

In some embodiments, L¹ is C₁₋₆-alkylene optionally substituted with one or more groups selected from oxo (═O), halogen, ═N and ═S. For example, L¹ may be substituted with one or more oxo group.

In some embodiments L¹ is —(C═O)C₁₋₄-alkylene-(C═O)—, e.g. L¹ is

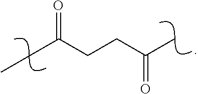

In some embodiments L¹ is absent.

In some embodiments, Z^{a} and L¹ are both absent.

In some embodiments R¹ is —NHC₁₋₁₈-alkyl, —NHC₆₋₁₆-Aryl, or —NH—C₁₋₆-alkyl-C₆₋₁₆aryl optionally substituted with one or more groups selected from halogen, C₁₋₆-alkyl, or C₁₋₆-haloalkyl. For example R¹ may be —NHC₆₋₁₆-Aryl optionally substituted with one or more groups selected from halogen, C₁₋₆-alkyl, or C₁₋₆-haloalkyl.

For example, R¹ may be selected from N(H)npropyl, —N(H)ipropyl, —N(H)nbutyl, —N(H)isoamyl, —N(H)nhexyl, —N(H)noctyl, —N(H)t-octyl, —N(H)ndecyl, —N(H)ndodecyl, or N(H)-fluorenyl optionally substituted with one or more halogen groups. For example, R¹ may be —N(H)-fluorenyl substituted with one or more bromine atom, e.g.:

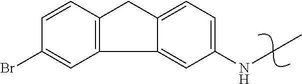 [SMILES: CCNc1ccc2c(c1)-c1cc(Br)ccc1C2]

For example, L¹-R¹ may be:

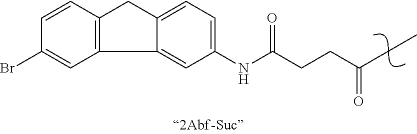 [SMILES: CCC(=O)CCC(=O)Nc1ccc2c(c1)-c1cc(Br)ccc1C2]

In some embodiments, R¹ is hydrogen (designated H or Hy; i.e. the compound has a free amine group at the N-terminus) or acetyl.

In some embodiments, R² is NH₂

The compound may be:

(SEQ ID NO: 18)
Hy-ASG[βhL]VAFPRV-NH₂ [2315];
(SEQ ID NO: 19)
Hy-ASGL[βhL]AFPRV-NH₂ [2316];
or
(SEQ ID NO: 20)
Hy-ASG[[βhL]V[[βA]FPRV-NH₂ [2320].

The invention further provides a composition, e.g. an insect control composition or plant protection composition, comprising a compound of the third aspect of the invention in admixture with one or more solvents, carriers, diluents, adjuvants, preservatives, dispersants, emulsifying agents, or synergists. The composition may be an aqueous composition.

The compounds of the third aspect have activity against hemipteran insects.

Also provided is the use, as an insect control agent, e.g. against hemipteran insects, of a compound of the third aspect of the invention.

The compounds typically increase insect mortality, in general, or under conditions of stress such as cold stress, desiccation stress or starvation stress. When used to increase insect mortality, the compounds described (and compositions containing them) may be regarded as insecticides. The compounds may also have activity in reducing insect fecundity, whether of individual insects or of an insect population as a whole. The effect on fecundity may be exerted in conjunction with an effect on mortality or independently thereof.

Without wishing to be bound by theory, any or all of the effects described may be mediated by binding of the compounds to the CAP2b receptor of the target hemipteran insects. The CAP2b receptor of M. persicae may be used as a model system, as described in the examples below. Thus, it may be preferable that the compounds have affinity for the CAP2b receptor, e.g. for the CAP2b receptor of M. persicae. In some embodiments, they have an agonistic effect on the CAP2b signalling pathway. In other embodiments, they may have an antagonistic effect on the CAP2b signalling pathway

The invention provides a method of increasing hemipteran insect mortality comprising contacting a hemipteran insect or hemipteran insect population with a compound as described.

The invention further provides a method of reducing cold tolerance, reducing desiccation stress tolerance, reducing starvation stress tolerance, and/or reducing fecundity of a hemipteran insect, or of a hemipteran insect population, comprising contacting a hemipteran insect or insect population with a compound as described.

The insect or insect population may be undergoing conditions of cold, desiccation stress, or starvation stress, as appropriate.

The compound may be applied directly to an insect or insect population. For example, it may be applied topically. Alternatively, the compound may be applied indirectly. For example, it may be applied to a substrate likely to come into contact with an insect or insect population. The substrate may be a plant, especially for Hemiptera which represent pests of plants (whether crops or horticultural plants). However, for Hemiptera which represent pests to humans, such as the Cimicidae family (e.g. bedbugs of the genus Cimex, such as Cimex lectularius) or the Reduviidae family (e.g. of the genus Rhodnius such as Rhodnius prolixus, or Triatoma such as Triatoma infestans) which can be vectors of human disease, the substrate may be a domestic surface or article, such as bedding, a mattress, or any other suitable domestic surface. The compound may be applied to the substrate in a form suitable for ingestion by an insect.

The invention further provides the use of a compound as described as a plant protection agent, and specifically for protecting a plant against hemipteran insects.

The invention further provides a method of inhibiting infestation of a plant by hemipteran insects comprising contacting the plant with a compound as described.

The method may be prophylactic. Thus, for example, the compound may be applied to the plant while the plant is free or substantially free of hemipteran insects.

Alternatively, the plant may already be colonised or infested by hemipteran insects. Thus, the invention further provides a method of reducing infestation of a plant, or of reducing hemipteran insect load on a plant, the method comprising contacting the plant with a compound as described.

In any of these embodiments, the compound may be provided as part of a composition, such as an insect control composition (e.g. insecticide composition) or a plant protection composition. Reference to application or use of a compound should therefore be construed as encompassing application or use of a suitable composition, unless the context demands otherwise.

The invention includes the combination of the aspects and preferred features described except where such a combination is clearly impermissible or expressly avoided.

### SUMMARY OF THE FIGURES AND TABLES

Table 1. The structure of biostable CAP2b, pyrokinin (with CAP2b receptor cross activity) and kinin analogues used in aphid stress tolerance assays. Modifications are shown in bold.

Table 2. The effect of neuropeptide analogue treatment via microinjection on the desiccation and starvation tolerance of M. persicae and M. rosae. Neuropeptide analogues were administered to a final concentration of ×10⁻⁵ M. Survival is shown as both a median survival (h)±IQR and an LTime50 (h). Values in bold significantly increased desiccation/starvation mortality in relation to a vehicle control group. Example survival curves are displayed in FIG. 1.

Table 3. Mean±SE of aphid life history traits when reared on a host plant, an artificial diet, or an artificial diet containing native CAPA neuropeptide, neuropeptide analogue 1895, or neuropeptide analogue 2129.

FIG. 1. Effect of CAP2b and kinin analogue treatment on the survival of Myzus persicae (1) and M. rosae (2) under conditions of desiccation and starvation stress. Control aphids are indicated by the black line and analogue-treated aphids by the blue line. CAP2b analogues 1895 (a) and 2129 (b) were administered to a final concentration of ×10⁻⁵ M via microinjection and acted to significantly increase mortality relative to the control. CAP2b analogue 2125 (c) and kinin analogue 2139 (d) are presented to illustrate non-significant survival curves.

FIG. 2. Survival curve calculated via Probit analysis of Myzus persicae pre-reproductive adults following a 1 hr exposure at the desired temperature. Raw data values are indicated by black circles.

FIG. 3. Mean±standard error proportion survival of M. persicae when treated with biostable peptide analogues (CAP2b/PK: 1895, 1896, 1902, 2089, 2123, 2125, 2129; kinin: 1728, 2139, 2139-Ac) via microinjection and subjected to a discriminating temperature for a 1 h exposure. Control groups are indicated by closed circle symbols and peptide treatment groups by open triangle symbols and dashed lines.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

Throughout the present description and claims the conventional three-letter and one-letter codes for naturally occurring amino acids are used, i.e.
A (Ala), G (Gly), L (Leu), I (Ile), V (Val), F (Phe), W (Trp), S (Ser), T (Thr), Y (Tyr), N (Asn), Q (Gin), D (Asp), E (Glu), K (Lys), R (Arg), H (His), M (Met), C (Cys) and P (Pro).

By “naturally occurring” in this context is meant the 20 amino acids encoded by the standard genetic code, sometimes referred to as proteinogenic amino acids.

Generally accepted three-letter codes and other abbreviations for other amino acids may also be employed, such as hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib), etc.

Such other amino acids may be shown in square brackets “[ ]” (e.g. “[Aib]”) when used in a general formula or sequence in the present specification, especially when the rest of the formula or sequence is shown using the single letter code.

Unless otherwise specified, amino acid residues in peptides of the invention are of the L-configuration. However, D-configuration amino acids may be incorporated. In the present context, an amino acid code written with a small letter may be used to represent the D-configuration of said amino acid.

Residues of beta amino acids may also be employed, particularly in compounds of the third aspect of the invention. Such residues may be designated by a “β” symbol followed by the conventional code for the corresponding alpha amino acid.

Thus [βhL] represents a residue of beta-homoleucine (3-amino-5-methylcaproic acid):

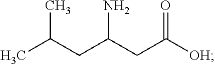

[βA] represents a residue of beta-alanine (3-aminopropanoic acid):

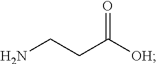

[βhA] represents a residue of beta-homoalanine:

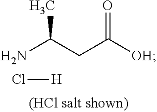

[βhV] represents a residue of beta-homovaline, sometimes referred to as beta-leucine (3-amino-4-methylpentanoic acid):

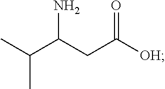

[βhF] represents a residue of beta-homo-phenylalanine:

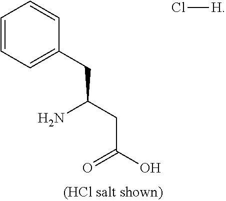

The notation Cₓ₋ₓₓ refers to the number of carbon atoms in a functional group. The number in the ‘x’ positions is the lowest number of carbon atoms and the number in the ‘xx’ position denotes the highest number of carbon atoms. For example, C₁₋₆-alkyl refers to an alkyl groups as defined herein having from 1 to 6 carbon atoms.

The notation i, n or t are used herein in relation to various alkyl groups in the normal way. Specifically, the suffixes refer to the arrangement of atoms and denotes straight chain (‘n’) or branched (‘i’ or ‘t’) alkyl groups.

The term alkyl as used herein refers to a saturated linear or branched-chain monovalent hydrocarbon radical, wherein the alkyl radical may be optionally substituted. The number of carbon atoms in the alkyl group may be specified using the above notation, for example, when there are from 1 to 8 carbon atoms the term “C₁₋₈-alkyl” may be used. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃).

The term alkylene as used herein refers to a saturated, branched, or straight chain hydrocarbon group having two monovalent radical centres derived by the removal of two hydrogen atoms from the same or two different carbon atoms of a parent alkane. The number of carbon atoms in the alkylene group may be specified using the above notation, for example, when there are from 1 to 8 carbon atoms the term “C₁₋₈-alkylene” may be used. Example alkylene groups include methylene (—CH₂—), 1,1-ethylene (—CH(CH₃)—), 1,2-ethylene (—CH₂CH₂—), 1,1-propylene (—CH(CH₂CH₃)—), 2,2-propylene (—C(CH₃)₂—).

The term alkenylene as used herein refers to a linear or branched-chain hydrocarbon group having two monovalent radical centres derived by the removal of two hydrogen atoms from the same or two different carbon atoms with at least one site of unsaturation, i.e., a carbon-carbon double bond. The alkenylene radical may be optionally substituted, and includes radicals having “cis” and “trans” orientations, or alternatively, “E” and “Z” orientations. The number of carbon atoms in the alkenylene group may be specified using the above notation, for example, when there are from 2 to 8 carbon atoms the term “Cm-alkenylene” may be used. Example alkenylene groups include, but are not limited to, ethenylene (—CH═CH—), prop-1-enylene (—CH═CHCH₂—),

In the chemical structures drawn herein, the presence of “ [symbol] ” denotes a point of attachment or a radical for example, a radical as discussed in relation to various functional groups. For example, the linker L¹ can optionally be:

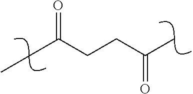

This structure has two points of attachment each denoted “ [symbol] ”. L¹ is attached to R¹ and Z^{a}. Thus R¹ may be attached at either of the attachment points, Z^{a} is then attached to the other attachment point.

The term aryl as used herein refers to a monovalent carbocyclic aromatic radical. Aryl includes groups having a single ring and groups having more than one ring such a fused rings or spirocycles. In the case of groups having more than one ring, at least one of the rings is aromatic. The number of carbon atoms in the aryl group may be specified using the above notation, for example, when there are from 6 to 16 carbon atoms the term “C₆₋₁₆-aryl” may be used. Aryl groups may be optionally substituted. Examples of aryl groups include phenyl, naphthyl, biphenyl, phenanthrenyl, naphthacenyl, 1,2,3,4-tetrahydronaphthalenyl, 1H-indenyl, 2,3-dihydro-1H-indenyl, and fluorenyl.

The term fluorenyl refers to the monovalent radical of the well known 3-fused ring core structure fluorene. Fluorene's structure is as follows:

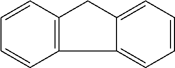

The term halogen as used herein refers the one or more of fluorine (F), chlorine (Cl), bromine (Br) or iodine (I).

The term haloalkyl refers to an alkyl group having on or more halogen substituent. The number of carbon atoms in the haloalkyl group may be specified using the above notation, for example, when there are from 1 to 8 carbon atoms the term “C₁₋₈-haloalkyl” may be used. Examples of haloalkyl groups include trifluoromethyl (—CF₃).

The term oxo (═O) as used here refers to a substituent. When an oxo group is present, the oxygen that makes the oxo group forms a double bond with the atom to which it is attached. For example, if a C₁-alkyl group (i.e. methyl) is substituted with an oxo group, a double bond is formed between the oxygen of the oxo group and the carbon of the methyl group, the resulting moiety is formyl (—(C═O)—).

The term acetyl (Ac) refers to:

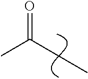

The term trifluoroacetyl refers to:

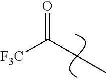

The term formyl refers to:

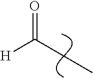

The term benzoyl refers to:

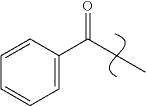

The benzoyl group may be optionally substituted.

The term (poly)alkyleneglycol means a moiety of the formula —O-(alkylene-O)ₙ— wherein ‘n’ is the number of alkyleneglycol units in the polymer, for example n may be from 1 to 50, preferably n is from 1 to 4, most preferably n is 1 or 2. Examples of (poly)alkyleneglycol groups include ethylene glycol (—O—CH₂—CH₂—O—), polyethylene glycol ((—O—CH₂—CH₂—O—), wherein n is an integer greater than 1 and propylene glycol (—O—CH₂—CH₂—CH₂—O—).

Terminal Groups L¹-R¹ and R²

The terminal groups present at the N- and C-termini of the peptide backbone are designated L¹-R¹ and R² respectively. Thus L¹-R¹ is bonded to the nitrogen atom of the N-terminal amino group and R² is bonded to the C-terminal carbonyl carbon atom.

L¹ may be absent. In such cases, R¹=“H” (or “Hy”; hydrogen) indicates a free primary amino group at the N-terminus. The other hydrogen atom of the N-terminal amino group is typically invariant, regardless of the nature of R¹, or L¹-R¹.

L¹

When present, L¹ is selected from C₁₋₆-alkylene, C₁₋₆-alkenylene and (poly)alkyleneglycol where each L¹ may be optionally substituted with one or more groups selected from oxo (═O), halogen, ═N and ═S.

In some embodiments L¹ is C₁₋₆-alkylene optionally substituted with one or more groups selected from oxo (═O), halogen, ═N and ═S. Preferably, L¹ is substituted with one or more oxo group.

In some embodiments L¹ is —(C═O)C₁₋₄-alkylene-(C═O)—, e.g. L¹ is

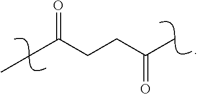

R¹

R¹ is hydrogen, C₁₋₄ alkyl, acetyl, formyl, benzoyl or trifluoroacetyl, —NHC₁₋₁₈-alkyl, —NHC₆₋₁₆-Aryl, or —NH—C₁₋₆-alkyl-C₆₋₁₀ aryl optionally substituted with one or more groups selected from halogen, C₁₋₆-alkyl, or C₁₋₆-haloalkyl.

In some embodiments, R¹ is —NHC₁₋₁₈-alkyl, —NHC₆₋₁₆-Aryl, or —NH—C₁₋₆-alkyl-C₆₋₁₆aryl optionally substituted with one or more groups selected from halogen, C₁₋₆-alkyl, or C₁₋₆-haloalkyl. For example, R¹ may be —NHC₆₋₁₆-Aryl optionally substituted with one or more groups selected from halogen, C₁₋₆-alkyl, or C₁₋₆-haloalkyl.

For example, R¹ may be selected from N(H)npropyl, —N(H)ipropyl, —N(H)nbutyl, —N(H)isoamyl, —N(H)nhexyl, —N(H)noctyl, —N(H)t-octyl, —N(H)ndecyl, —N(H)ndodecyl, or N(H)-fluorenyl optionally substituted with one or more halogen groups. Preferably, R¹ is —N(H)-fluorenyl substituted with one or more bromine atom, e.g. R¹ is:

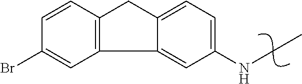 [SMILES: CCNc1ccc2c(c1)-c1cc(Br)ccc1C2]

L¹ and R¹

In some embodiments:

(i) L¹ is C₁₋₆-alkylene optionally substituted with one or more groups selected from oxo (═O), halogen, ═N and ═S; e.g. L¹ is substituted with one or more oxo group; and
(ii) R¹ is —NHC₁₋₁₈-alkyl, —NHC₆₋₁₆-aryl or —NH—C₁₋₆-alkyl-C₆₋₁₆aryl, optionally substituted with one or more groups selected from halogen, C₁₋₆-alkyl, or C₁₋₆-haloalkyl; e.g., R¹ is —NHC₆₋₁₆-aryl optionally substituted with one or more groups selected from halogen, C₁₋₆-alkyl, or C₁₋₆-haloalkyl.

In some embodiments:

(i) L¹ is C₁₋₆-alkylene optionally substituted with one or more groups selected from oxo (═O), halogen, ═N and ═S; and
(ii) R¹ is selected from N(H)npropyl, —N(H)ipropyl, —N(H)nbutyl, —N(H)isoamyl, —N(H)nhexyl, —N(H)noctyl, —N(H)tert-octyl, —N(H)ndecyl, —N(H)ndodecyl or N(H)-fluorenyl, optionally substituted with one or more halogen groups.

In some embodiments:

(i) L¹ is C₁₋₆-alkylene substituted with one or more oxo (═O) groups; and
(ii) R¹ is N(H)-fluorenyl optionally substituted with one or more halogen groups, e.g. one or more bromine groups.

In some embodiments L¹ is

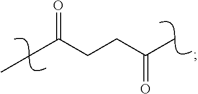

and

R¹ is

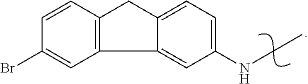 [SMILES: CCNc1ccc2c(c1)-c1cc(Br)ccc1C2]

For example, L¹-R¹ may be:

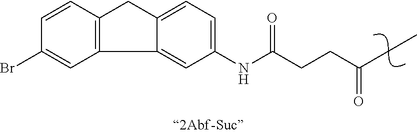 [SMILES: CCC(=O)CCC(=O)Nc1ccc2c(c1)-c1cc(Br)ccc1C2]

R²

R² is “—OR2^{a}” or “—NH₂”, indicating a C-terminal ester (COOR²a) or amido (CONH₂) group respectively. Typically, R₂ is NH₂.

Peptide Z^{a}

Za is an optional additional peptide sequence of 1 to 8 amino acids in length. Thus it may be 1, 2, 3, 4, 5, 6, 7, or 8 residues in length.

Without wishing to be bound by theory, it is believed that longer Z^{a} sequences may introduce too great a distance between the peptide Z^{a} and the R¹ group, leading to sub-optimal receptor interaction.

Z^{a} may contain one or more non-proteinogenic amino acids. For example, it may contain one of more beta amino acids, or one or more D-amino acids.

In some embodiments, it may be desirable that Z^{a} is composed primarily or entirely of small residues such as Ser, Gly and Ala, e.g. at least half of the residues in Z^{a} may be selected from Ser, Gly and Ala.

Insect Control Agent

The term “insect control agent” refers to agents when used to increase mortality (i.e. as insecticides) and/or when used to reduce fecundity. Thus an insect control agent may be administered to accelerate mortality of a given insect or insect population, to reduce fecundity of a given insect or insect population.

Without wishing to be bound by theory it is believed that certain compounds described in this specification are able to reduce the reproductive lifetime (i.e. days as a reproducing adult), the rate of reproduction (number of offspring produced per day as reproducing adult) and/or the total lifetime progeny of treated insects. Thus any or all of these factors can be taken into account when assessing effect on fecundity.

Whatever the mechanism involved, an insect control agent may be used to reduce the size of an insect population, or inhibit growth of an insect population (e.g. as compared to an otherwise identical insect population not exposed to the agent).

An insect control composition is a composition comprising an insect control agent as described.

Plant Protection Agent

The term “plant protection agent” refers to agents when used to protect a plant against hemipteran insects, e.g. against infestation or colonisation, or being used as a food source by such insects (e.g. by the draining of sap). Infestation or colonisation may be by larvae (or nymphs), by adult insects, or by being used as a host or repository for eggs. The terms “infestation” and “colonisation” should not be construed as requiring the presence of the insects to be deleterious to the plant, however.

A plant protection agent may be applied inter alia for reducing insect load on a plant, for inhibiting (e.g. reducing the rate of) increase of insect load on a plant, or for maintaining a plant in an insect-free state. Thus, the agent may be applied to a plant which already carries hemipteran insects, or to a plant which is free or substantially free of hemipteran insects.

A plant protection composition is a composition comprising an plant protection agent as described.

Effect on Insect Mortality Under Stress Conditions

Insects are ectotherms with high surface area to volume ratios; maintaining water balance and tolerating temperature fluctuations thus are essential adaptations. In effect, most insects live under an almost constant state of desiccation stress. A key mechanism used by insects to maintain water balance is to reduce the rate of water loss. In low temperature environments insects face both chilling and low availability of water, thus requiring that they be both cold and desiccation tolerant. Insect cold tolerance is increasingly of interest as invasive insect species expand their geographical range, as this often requires adaptation to colder zones and ability to tolerate colder climates. Both cold and desiccation stress result in decreased hemolymph volume and increased hemolymph osmolarity. Capa and kinin peptides have previously been shown to regulate or modulate insect responses to desiccation and cold stress. see, for example, Terhzaz et al., (2015).

Certain of the compounds described in this specification are able to increase insect mortality under stress conditions, e.g. under conditions of cold stress. In addition, certain compounds also increase mortality under conditions of starvation stress, and/or are able to reduce the reproductive lifetime (i.e. days as a reproducing adult), the rate of reproduction (number of offspring produced per day as reproducing adult) and/or the total lifetime progeny of treated insects. Compound 1895 and 2129 are particularly effective in this context. Thus the compounds find use as insecticides against hemipteran insects, particularly against hemipteran insects likely to be experiencing cold and/or starvation stress.

Still others are able to increase mortality in the absence of additional stress conditions, such as 2135, 2136 and 2320.

Additionally or alternatively, as already described, certain of the compounds find use as plant protection agents or insect control agents, independently of any effect on insect mortality, via their effect on reproductive lifetime and fecundity.

Hempiteran Insects

The compounds and compositions of the invention have activity against insects of the Order Hemiptera, which comprises groups including aphids, planthoppers, leafhoppers, stink bugs, shield bugs and cicadas.

Hemipterans are defined by distinctive mouthparts in the form of a “beak”, comprising modified mandibles and maxillae which form a “stylet”, sheathed within a modified labium.

Many insects within these groups have endogenous neuropeptides with sequence homology to the analogues 1895 and 2129, suggesting that these analogues may have activity against those insects.

The insects may belong to the sub-order Sternorrhyncha, e.g. to the super-family of Aphidoidea (aphid superfamily), Aleyrodoidea (whiteflies), Coccoidea (scale insects), Phylloxeroidea (including Phylloxeridae or “phylloxerans”, and Adelgidae or woolly conifer aphids) or Psylloidea (jumping plant lice etc.).

Thus, the insects may be aphids, i.e. members of the aphid superfamily (Aphidoidea). Aphids (Hemiptera: Aphididae) are one of the most significant groups of agricultural pests³⁸ and are vectors in the transmission of approximately 50% of all insect transmitted plant viruses.³⁹ Within that superfamily, the aphids may be part of the family Aphididae, which contains sub-families Aiceoninae, Anoeciinae, Aphidinae, Baltichaitophorinae, Calaphidinae, Chaitophorinae, Drepanosiphinae, Eriosomatinae, Greenideinae, Hormaphidinae, Israelaphidinae, Lachninae, Lizeriinae, Macropodaphidinae, Mindarinae, Neophyllaphidinae, Phloeomyzinae, Phyllaphidinae, Pterastheniinae, Saltusaphidinae, Spicaphidinae, Taiwanaphidinae, Tamaliinae and Thelaxinae.

The secondary study species, the rose aphid Macrosiphum rosae, was selected to represent a major pest of horticulture. M. rosae is an important pest of cultivated species of Rosa and is a vector in the transmission of 12 plant viruses including the strawberry mild yellow edge virus.⁴¹

The aphids may, for example, be of the genus Acyrthosiphon (e.g. Acyrthosiphon pisum), Aphis (e.g. Aphis gossypii, Aphis glycines), Diuraphis (e.g. Diuraphis noxia) Macrosiphum (e.g. Macrosiphum rosae, Macrosiphum euphorbiae), Myzus (e.g. Myzus persicae), or Sitobion (e.g. Sitobion avenae).

Myzus persicae (peach potato aphid) is the most economically important aphid crop pest worldwide,⁴⁰ with a global distribution and host range encompassing more than 400 species in 40 different plant families.⁴¹ For example, it is a major pest of agricultural crops including fruit and potatoes, and act as a vector for viruses.

Macrosiphum rosae, (rose aphid) is an important horticultural pest, especially of cultivated species of Rosa, and is a vector in the transmission of 12 plant viruses including the strawberry mild yellow edge virus.⁴¹

Aphis gossypii (cotton or melon aphid) is a pest of Curcibitae and cotton.

Other than aphids, the insects may, for example, be of the Adelgidae family, e.g. of the genus Adelges (e.g. Adelges tsugae).

The insects may be of the Aleyrodidae family, e.g. of the genus Bemisia (e.g. Bemisia tabaci) or Trialeurodes (e.g. Trialeurodes vaporariorum).

The insects may be of the Psylloidea family, e.g. of the genus Pachypsylla (e.g. Pachypsylla venusta).

As examples of hemipteran insects outside the sub-order Sternorryncha, the insects may be of the Cimicidae family, e.g. of the genus Cimex (bed bugs), e.g. Cimex lectularius.

The insects may be of the Cicadellidae family, e.g. of the genus Cuerna (e.g. Cuerna arida), Graminella (e.g. Graminella nigrifrons) or Homalodisca (e.g. Homalodisca vitripennis).

The insects may be part of the Delphacidae family, e.g. of the genus Nilaparvata (e.g. Nilaparvata lugens) or Sogatella (e.g. Sogatella furcifera). For example, Nilaparvata lugens (brown planthopper) is a pest of rice crops, especially in Asia.

The insects may be of the Liviidae family, e.g. of the genus Diaphorina (e.g. Diaphorina citri).

The insects may be part of the Miridae family, e.g. of the genus Pseudatomoscelis (e.g. Pseudatomoscelis seriatus), Lygus (e.g. Lygus hesperus) or Tupiocoris (e.g. Tupiocoris notatus). For example, Pseudatomoscelis seriatus (cotton fleahopper) is a pest of cotton.

The insects may be of the Pentatomidae family, e.g. of the genus Acrosternum (e.g. Acrosternum hilare), Banasa (e.g. Banasa dimiata), Euschistus (e.g. Euschistus servus), Halyomorpha (e.g. Halyomorpha halys), Murgantia (e.g. Murgantia histrionica), Nezara (e.g. Nezara viridula), Plautia (e.g. Plautia stali), or Podisus (e.g. Podisus maculiventris). For example, Acrosternum hilare (green stink bug) is a significant pest of cotton. Euschistus servus (brown stink bug) is a pest of many agricultural crops including seeds, grains, nuts and fruits, especially in the southern USA. Nezara viridula is a pest of grain and soybean crops, especially in Brazil.

The insects may be of the Pyrrhocoridae family, e.g. of the genus Pyrrhocoris (e.g. Pyrrhocoris apterus).

The insects may be of the Reduviidae family, e.g. of the genus Rhodnius (e.g. Rhodnius prolixus), or Triatoma (e.g. Triatoma infestans). Rhodnius prolixus is a vector of human disease (Chagas disease).

The insects may be of the Triozidae family, e.g. of the genus Acanthocasuarina (e.g. Acanthocasuarina muellerianae).

Compositions

Compositions of the invention, or for use in accordance with the invention, typically comprise a compound as described in combination with one or more ancillary component such as solvents, carriers, diluents, adjuvants, preservatives, dispersants, emulsifying agents, or synergists.

The composition may be an aqueous composition, e.g. a saline composition. The aqueous composition may contain one or more buffers, such as a phosphate buffer (e.g. phosphate buffered saline) or a Tris buffer. Alternatively the composition may be an oil dispersion or an emulsion, e.g. an oil and water emulsion.

Adjuvants may enhance product performance, for example, by increasing efficiency of delivery of active ingredients, reducing the level of active ingredient required, or extending the spectrum of effectiveness.

Different types of adjuvants offer various benefits and advantages, which are achieved by modulating properties such as spray formation, spray retention, wetting, deposit formation or uptake.

Adjuvants modulating spray formation may influence spray quality by reducing spray drift and wastage, allowing more of the product to reach the target. This can reduced use rates, leading to a better environmental profile and a potentially more cost effective solution. Such adjuvants include non-ionic surfactants and emulsifier blends.

Adjuvants modulating spray retention may dissipate the kinetic energy of the droplet during impact, meaning the likelihood of bounce or run-off is reduced. Such adjuvants include alkyl polyglucosides, alkoxylated alcohols, and polyoxyethylene monobranched alcohols (e.g. polyoxyethylene (8) monobranched alcohol).

Adjuvants modulating wetting properties (i.e. wetting agents) may reduce surface tension and contact angle, leading to enhanced coverage. Such adjuvants include polyoxyethylene sorbitan monolaurate (e.g. polyoxyethylene (8) sorbitan monolaurate), surfactant blends, and alkyl polyglucosides.

Adjuvants modulating deposit formation may influence evaporation of water from the droplet and thus provide a more homogeneous distribution. Such adjuvants include alkoxylated polyol esters, polyoxyethylene sorbitan monolaurate (e.g. polyoxyethylene (12) sorbitan monolaurate), and alkyl polyglucoside.

Adjuvants modulating uptake can improve penetration and uptake of active ingredients. e.g. through the insect cuticle, resulting in increased bioavailability. Such adjuvants include alkoxylated polyol esters and polyoxyethylene sorbitan monolaurate (e.g. polyoxyethylene (12) sorbitan monolaurate and polyoxyethylene (16) sorbitan monolaurate).

Dispersants may be aqueous or non-aqueous. An oil dispersion (OD) formulation typically comprises a solid active ingredient dispersed in oil. The oil can vary from paraffinic to aromatic solvent types and vegetable oil or methylated seed oils. Typically the active ingredient is uniformly suspended in the oil phase. Although primarily used for water sensitive active ingredients, OD formulations have extended to other active ingredients due to their better spray retention, spreading, foliar uptake, and penetration enhancement (e.g. across the insect cuticle) as the carrier oil often acts as an adjuvant.

Oils suitable for use in OD dispersions include linseed, rapeseed and soyabean oils.

Aqueous dispersants may be used, for example, to improve stability in the spray tank after dilution in water, and may include modified styrene acrylic polymers, and polymeric amphoteric dispersants and adjuvants.

An emulsifier may be employed to emulsify a continuous oil phase into water when an OD formulation is diluted prior to being sprayed. The emulsifier may be selected based upon their ability to spontaneously form the emulsion. Their performance is primarily dictated by the nature of the surfactant and their collective effect on how they arrange themselves at the oil/water interface. Examples include polyoxyethylene sorbitol hexaoleate (e.g. polyoxyethylene (40) sorbitol hexaoleate), emulsifier blends, and calcium alkylaryl sulphonate.

The compound may be provided in the form of a concentrate, for dilution prior to application. Alternatively the compound may be provided in a solid form to be suspended or dissolved prior to formulation.

The composition may be a bait composition for ingestion by the target insect. A bait composition may comprise one or more phagostimulants, i.e. a substance which will entice the insect to ingest the compound. Phagostimulants may include artificial sweeteners, amino acids, other peptides or proteins and carbohydrates (e.g. glucose, fructose, sucrose, maltose) etc. Examples include honey, syrups and aqueous solutions of sucrose.

Commercially available base formulations may also be suitable for use in formulating the compounds described in this specification, such as Armid® FMPC (Akzo Nobel).

The composition may comprise one or more synergists, i.e. compounds which increase the efficacy of insecticides against their targets, often by inhibiting an insect's ability to metabolise the active agent. Common synergists include piperonyl butoxide and MGK-264 (n-octyl bicycloheptane dicarboximide).

The composition may further comprise one or more additional active insecticides, such as (but not limited to) pyrethrins or pyrethroids. The choice of ancillary or additional insecticides will typically depend on the particular target species.

### EXAMPLES

Materials and Methods

Aphid Rearing

Stock cultures of anholocyclic M. persicae were established using aphids supplied by the Smagghe laboratory, Ghent University, Belgium. Cultures were reared under a 12:12 h LD photocycle at 22° C. on Chinese cabbage (Brassica rapa var. Wong Bok) contained within a BugDorm fine mesh cage (44545F) (45 cm×45 cm×45 cm). A fresh supply of Chinese cabbage of approximately 4 weeks from sowing was supplied to the cages on a once-weekly basis to maintain the aphid cultures.

M. rosae was selected as a secondary aphid species and a sub-set of experiments was performed on the species to determine the overlap in response between aphid species of different genera. Stock cultures of anholocyclic M. rosae were set up from individual aphids originally collected on Rosa species within the grounds of the University of Glasgow, Scotland, UK. A stock culture was set up within the laboratory and maintained on shop bought miniature rose plants and under identical conditions to M. persicae.

Peptide Synthesis

Native and fluorescently labelled neuropeptides CAPA-1 and kinin were synthesized by Cambridge Peptides (Birmingham, UK) as previously detailed⁷ and based on the CAPA and kinin structures of Drosophila melanogaster. In brief, native kinin was synthesized and coupled to Alexfluor488 resulting in fluorescent kinin (Alexa-488-C₅-maleimide-CNSVVLGKKQRFHSWGamide (SEQ ID NO: 21)). The same rationale was used for the production of CAPA-1 (GANMGLYAFPRVamide (SEQ ID NO: 22)) and labelled CAPA-1 with the addition of TMR-C₅-Maleimide Bodipy dye (TMR-C₅-maleimide-CGANMGLYAFPRVamide (SEQ ID NO: 23)).

The synthesis of PK analogues (with CAP2b receptor cross activity) 1895 and 1902,^{22,23} CAP2b analogue 1896,²² and insect kinin analogues 1728 and 2139^{29,30} have been previously described. CAP2b analogues 2089, 2123, 2125, and 2129;²³ as well as insect kinin analogue 2139-Ac²⁹ were synthesized and cleaved according to procedures that have been previously described. The analogues were purified on a Waters Delta-Pak C18 reverse-phase column (8×100 mm, 15 μm particle size, 100 Å pore size) with a Waters 510 HPLC system with detection at 214 nm at ambient temperature. Solvent A=0.1% aqueous trifluoroacetic acid (TFA); Solvent B=80% aqueous acetonitrile containing 0.1% TFA. Initial conditions were 10% B followed by a linear increase to 90% B over 40 min.; flow rate, 2 ml/min. Delta-Pak C18 retention times: 2089, 12.0 min.; 2123, 9.0 min; 2139-Ac, 5.9 min; 2125, 12.5 min; 2129, 7.5 min. The analogues were further purified on a Waters Protein Pak I 125 column (7.8×300 mm). Conditions: isocratic using 80% acetonitrile containing 0.1% TFA; flow rate, 2 ml/min. Waters Protein Pak retention times: 2089, 6.0 min; 2123, 5.5 min; 2139-Ac, 5.9 min; 2125, 5.5 min; 2129, 6.0 min. Amino acid analysis was carried out under previously reported conditions (Nachman et al., 2004) to quantify the analogues and to confirm identity: 2089: F[1.0], P[1.0], R[1.0], T[1.0], V[1.0]; 2123: F[1.0], R[0.9], T[0.9], V[0.9]; 2139-Ac: F[2.0], G[0.9]; 2125: F[1.0], R[0.8], T[0.7], V[0.8]; 2129: A[1.0], I[0.9], P[0.9], R[0.9], T[0.9]. The identity of the analogues was also confirmed by MALDI-MS on a Kratos Kompact Probe MALDI-MS instrument (Shimadzu, Columbia, Md.). The following molecular ions (MH+) were observed: 2089, 961.0 (calc.961.8); 2123, 976.2 (calc. 976.1); 2139-Ac, 704.7 (calc. 704.5, [MNa+]); 2125, 1014.1 (calc. 1014.0); 2129, 898.8 (calc. 898.8). The structures of the biostable analogues are displayed in Table 1.

Receptor Mapping Assay Using Fluorescently Labelled Neuropeptides

Aphids were cold anesthetized and the tissue of interest dissected out in a 1:1 solution of Schneider's insect medium and optimized saline.^{7,42} The dissected tissue was mounted on a poly-L-lysine-covered 35 mm glass bottom dish containing 1:1 saline. Nuclei were stained via incubation in DAPI (1 μg ml⁻¹) and a baseline image taken to determine the level of autofluorescence and adjust exposure settings accordingly. All images were recorded on an inverted confocal microscope (Zeiss LSM 510 Meta). A labelled neuropeptide (10⁻⁷ M) was subsequently added and the sample imaged. The concentration of 10⁻⁷ M was chosen for labelled neuropeptides because it represents the minimal concentration required to produce a saturated receptor response, thereby optimizing the conditions for optical detection of ligand-receptor complexes.⁷ Following imagining, unlabelled neuropeptide (10⁻⁵ M) was added to the sample and a time-lapse experiment set up to determine if the unlabelled neuropeptide outcompeted the labelled neuropeptide, thus reaffirming the detection of the ligand-receptor complexes. Images were collected every 30 s for a duration of 20-30 m. All images were exported as JPEG files and subsequently viewed in FIJI and Microsoft Illustrator. When specific binding was observed in muscle tissue, this was supported by the addition of rhodamine phalloidin; a high-affinity F-actin probe conjugated to tetramethylrhodamine (TRITC) that specifically binds to muscle.

Peptide Treatment Via Microinjection

Neuropeptides were administered to test aphids via microinjection to allow for rapid mass screening of neuropeptide analogue efficacy. For this, native neuropeptides were diluted in double distilled water (DDH₂O) to a concentration of 1×10⁻⁵ M. Neuropeptide analogues were diluted in DDH₂O to the following concentrations: kinin analogues 1728 (2.5×10⁻⁵ M), 2139 (3.5×10⁻⁵ M), 2139-Ac (3.5×10⁻⁵ M); CAP2b analogues 1895 (3.5×10⁻⁵ M), 1896 (3.5×10⁻⁵ M), 1902 (3.5×10⁻⁵ M), 2089 (3.9×10⁻⁵ M), 2123 (1.0×10⁻⁵ M), 2125 (1.0×10⁻⁵ M), 2129 (2.0×10⁻⁵ M). Once at the desired concentration, neuropeptide solutions were administered to test aphids at an injection volume of 9 nl based on total haemolymph volume, to produce an approximate 1:20 dilution of injection volume to haemolymph. Injections were performed using a pulled glass needle and a Nanoject II Auto-Nanoliter Injector (Drummond Scientific Company, Broomall, Pa.). A vehicle control was set up for each treatment/day of experiments to account for variation in needle pulling. For this, control aphids were injected with 9 nl of DDH₂O and subsequently exposed to the same experiments as aphids receiving the neuropeptide treatment. Neuropeptide treated and vehicle control aphids were subsequently used in the stress bioassays detailed below.

For peptides 2315, 2320 and 2125, peptides were diluted individually in an Armid FMPC formulation (AkzoNobel Surface Chemistry, Stenungsund, Sweden) to concentration of 1×10⁻⁵ M. Using a Nanoject II Auto-Nanoliter micro-injector, 9 nl of the peptide solution was applied topically to the abdomen of a pre-reproductive adult aphid, coating the cuticle in the solution. Aphids were returned to the host plant and allowed to recover for 24 h before use in experiments. Control aphids were topically applied with 9 nl of the Armid FMPC formulation and, once again, allowed to recover for 24 h on the host plant before use in experiments.

Desiccation/Starvation Tolerance Assay

Anholocyclic adults of mixed age of either M. persicae or M. rosae were selected from the stock cultures and treated with a native neuropeptide or neuropeptide analogue via microinjection using the method detailed above. Treated and vehicle control aphids were allowed to recover on excised leaves of the host plant for 1 h before being placed in an empty ventilated microcage (L=4 cm, 0=9.5 cm) at densities of 10 per cage. In total, 30-40 aphids were treated for each neuropeptide treatment group and a further 30-40 for the associated vehicle control group. From the point of placement in the microcage (taken as 0 h), aphid survival was checked every hour during daylight hours and approximately every 4 h during night-time hours until the final aphid died. Survival data were subsequently analysed using a Log-rank (Mantel-Cox) text in GraphPad Prism version 7.0. LTime₅₀ (the time taken to kill 50% of the test population) values were calculated via Probit Analysis in Minitab 17 (Minitab Inc., State College, Pa.).

Cold Tolerance Bioassay

Calculation of Discriminating Temperatures

M. persicae and M. rosae displayed identical results in desiccation/starvation stress assays. For this reason, and given its global pest status, only M. persicae was taken forward in cold stress assays. Survival curves were first established to determine a species-specific discriminating temperature for subsequent neuropeptide testing. Aphids were selected at the pre-reproductive adult stage for cold tolerance bioassays since aphid cold tolerance is known to significantly vary throughout an aphid's life cycle.^{43,44} Temperature ranges were selected to encompass 0-100% mortality. Anholocyclic pre-reproductive adults (approximately 9 d old at 22° C.) of M. persicae were exposed to a range of low temperatures (−14° C. to −7° C. at 1° C. intervals) using a direct plunge method.^{45,46} For each temperature treatment, 30 adults were placed within plastic 0.5 mL Eppendorf tubes at densities of ten adults per tube, which, in turn, were placed within a glass boiling tube held within an alcohol bath (Haake G50 and PC200; Thermo Scientific, Germany) pre-set to the desired temperature. Pieces of cotton wool were used to stopper the boiling tubes to limit air circulation and to ensure a more stable internal temperature within the tubes. Adults were held at the desired exposure temperature for 1 h. Following exposure, aphids were allowed to recover at the culture temperature in microcages containing excised leaves of the host plant and survival was assessed after 48 h. The procedure was repeated for each exposure temperature.

Survival data were analysed using Probit analysis in MINITAB, version 17 (Minitab Inc., State College, Pa.) and the LT₃₀ (the lethal temperature resulting in 30% mortality of a test population) was elucidated. The LT₃₀ was chosen to act as a discriminating temperature for subsequent neuropeptide testing since it enabled detection of directional effects of subsequent neuropeptide treatment, but primarily in the direction of interest i.e. which neuropeptides significantly increased mortality in the species of interest.

Peptide Analogue Treatment and Testing at the Discriminating Temperature

Pre-reproductive anholocyclic adult aphids of M. persicae were treated with neuropeptide analogues using the microinjection method detailed above. Following microinjection treatment, individuals were returned to microcages containing excised leaves of the host plant at densities of approximately 20-30 per microcage and allowed to recover for 24 h at the culture temperature. Following the 24 h recovery period, adults were placed within plastic 0.5 mL Eppendorf tubes at densities of ten adults per tube to a total of 30 for each species x neuropeptide treatment group. Eppendorf tubes were then placed within glass boiling tubes held within the alcohol bath pre-set to the desired discriminating temperature. Pieces of cotton wool were used to stopper the boiling tubes to limit air circulation and to ensure a more stable internal temperature within the tubes. Adults were held at the desired exposure temperature for 1 h. Following exposure, adults were allowed to recover at the culture temperature in microcages containing excised leaves of the host plant and survival was assessed after 48 h. The procedure was repeated for each species x peptide analogue treatment group.

Statistical analyses were performed using R Software (R Development Core Team, 2013). A generalised linear model (GLM) with binomial family was fitted to survival data with analogue ‘Treatment’ (peptide analogue), treatment ‘Type’ (test vs. control), and analogue treatment x treatment type interaction as factors.

Feeding of Aphids with Peptides in Artificial Diet; Effects on Mortality, Life Span and Fecundity

A standard artificial diet for M. persicae was produced as described in Van Emden (2009) and provided the basal diet to which neuropeptide analogues were added for screening purposes. Neuropeptide analogues were diluted individually in the artificial diet to a pre-determined recommended concentration as follows: 1895 (3.5×10⁻⁵ M) and 2019 (2.0×10⁻⁵ M).

Feeding apparatus were constructed using a set-up developed by Sadeghi et al (2009). For this, a piece of Parafilm was stretched over a Plexiglas ring (h=4 cm, Ø=3 cm) and 100 μl of the artificial diet containing the desired neuropeptide analogue was pipette onto the Parafilm membrane. A second piece of Parafilm, stretched to 4 times the original thickness, was stretched over the original layer, sandwiching the artificial diet between two layers of Parafilm. A strip of Parafilm was wrapped around the circumference of the Plexiglas ring, sealing in the diet. A plastic ring (h=1.2 cm, Ø=3.4 cm) was subsequently placed over the Parafilm layer, creating a walled chamber in which to house test aphids in contact with the Parafilm layer containing the artificial diet. Finally, a small Petri dish (h=1 cm, Ø=3.6 cm), modified for ventilation with net cloth, was placed on top of each feeding apparatus to prevent aphid escape.

To obtain aphids for use in experiments, reproducing anholocyclic adults were placed on individual excised leaves of Chinese cabbage at densities of 5 adults per leaf and allowed to reproduce for 24 h. The stem of each excised leaf was held within a 0.5 mL Eppendorf tube containing water via a punctured hole in the Eppendorf lid, and placed individually within a microcage (L=4 cm, Ø=9.5 cm). Following 24 h, adults were removed and resultant first instar nymphs (<1 day old and synchronised in age to within 24 h) retained. Nymphs were allowed to develop on the Chinese cabbage for 5 days. On day 5, (3^{rd} instar) nymphs were transferred onto the artificial diet containing a neuropeptide analogue at densities of 1 per feeding chamber and monitored daily until death. Aphids were transferred to fresh artificial diet (containing neuropeptide analogue) every 5 days. Life history traits recorded include age at first reproduction, number of nymphs produced per 24 h period, and lifespan. From these parameters, lifetime fecundity and daily fecundity were calculated. Control groups were set up involving aphids reared on the host plant, an artificial diet without a neuropeptide analogue, and an artificial diet containing the native CAPA neuropeptide.

Topical Application by Spraying

Aphids were exposed to test peptides in the absence of additional external stress conditions.

Air Brush

Brassica rapa (Chinese cabbage; Wong Bok) were infested with 30 adult Myzus persicae aphids per plant. Aphids were left at least 2 hours to settle and begin feeding from the host plant.

Spraying took place inside an externally vented fume cupboard. No mist or vapours were observed to escape from the fume cupboard during the course of spraying. To ensure spray tracking, all sprayed solutions had amaranth dye added, allowing the full surface of the plant to be evenly coated with the aerosolised compound.

The plants to be sprayed were placed inside a plastic disposal bag, inside the fume cupboard. This was used to control the area exposed to the aerosolised liquid, to avoid extensive spraying and cleaning inside the fume cupboard. The bag was further lined with absorbent paper towel to again help contain and control spray.

Each experiment had three controls:

i) no vehicle or peptide air spray only, negative control to assess effect of spraying on aphid ability to remain attached to the plant;
ii) vehicle spray only (Tween 24 0.1%); and
iii) Imidacloprid positive control (28.3 μM).

Imidacloprid was always applied last to prevent any possibility of stray pesticide being left inside the disposal bag and contaminating a test peptide applied plant.

Spray volumes for all solutions for this experiment 750 μl.

A clean air brush (ABEST AC06k30) was loaded with 750 μl of peptide solution diluted in Tween 24 0.1% vehicle. For this experiment there were three test conditions: 1895 alone (1×10⁻⁵M); 2129 alone (1×10⁻⁵M); and a simultaneous co-application of 1895+2129 together (1×10⁻⁵M of each peptide). During application, the air brush was held within the plastic bag and the compressor turned on. The air brush was then gently sprayed back and forth across the plant. To ensure 100% coverage of the sprayed liquid across the plant, the plant was held and physically rotated and moved to bring unsprayed sections into view. Care was taken to ensure peptide was sprayed across the upper and under side of the leaves, and around the stem. Application continued until the liquid loaded in to the air brush was exhausted or the plant was completely saturated with liquid. Distance between the plant and air brush was kept as constant as possible for a hand held device.

During this spray process, due to the pressure of the air stream and, at times, necessity of holding the air brush close to the plant, loosely attached aphids could become displaced. When this was observed they were placed back on the plant post spraying with a paint brush. Any aphid damaged by this recovery process was discarded.

Post peptide application, each condition was placed into its own individual Bugdorm (Watkins and Doncaster, 44545), to prevent repulsed or displaced aphids moving from one condition to another. Numbers of alive and dead aphids on the plant were counted 48 hours post spray, and the presence of any fresh nymphs noted. Plants were watered prior to spraying but not afterwards to eliminate the possibility this would drown any aphids present or wash off the sprayed liquid.

The air brush was cleaned in between each use of peptide.

Potter Spray Tower

Brassica rapa (Chinese cabbage; Wong Bok) were infested with 30 adult Myzus persicae aphids per plant. Aphids were left at least 2 hours to settle and begin feeding from the host plant.

Spraying took place inside a designated spray room. To ensure spray tracking, all sprayed solutions had amaranth dye added.

Potter Spray Tower (Burkard Manufacturing) was ‘primed’ by spraying 1000 μl of liquid coating the inside of the tower. Two controls were used:

i) vehicle spray only (Croda ATPlus UEP 100 LQ-(CQ) 0.1% v/v); and
ii) Imidacloprid positive control (28.3 μM).

Imidacloprid was always applied last and via a second, separate, Potter Tower, to prevent any possibility of stray pesticide being left inside the tower and contaminating a test peptide-applied plant.

Spray volumes for all solutions were 3000 μl. The 6.9 mm spray head was loaded with 3000 μl of a 1×10⁻⁵M peptide solution diluted in ATPlus 0.1%. After spraying was completed the plant was allowed to rest on the spray platform for 30 seconds to allow settling of the sprayed chemical.

For this experiment there were three test conditions: 1895 alone (1×10⁻⁵M); 2129 alone (1×10⁻⁵M); and a simultaneous co-application of 1895+2129 together (1×10⁻⁵M of each peptide).

During this spray process, due to the low pressure of the air stream, no aphids were observed to be dislodged from the plant.

Post peptide application, each condition was placed into its own individual Bugdorm (Watkins and Doncaster, 44545), to prevent repulsed or displaced aphids moving from one condition to another. Numbers of alive and dead aphids on the plant were counted 48 hours post spray, and the presence of any fresh nymphs noted. Plants were watered prior to spraying but not afterwards to eliminate the possibility of drowning any aphids present or washing off the sprayed liquid.

Post spraying, the spray head was filled with over 3000 μl of 70% ethanol and sprayed until empty. The spray head was carefully removed and rinsed with 70% ethanol as some amaranth dye was observed on the spray head. The inside of the tower was further cleaned by spraying 70% ethanol around the top and allowing it to drain down inside. The tower was then cleaned thoroughly by passing blue roll down from the top and up from the bottom of the tower. The spray platform is temporarily removed to allow access. The Potter Towers are cleaned between each use of peptide and at the end of experiments.

Results

Receptor Mapping Assay Using Fluorescently Labelled Neuropeptides

A fluorescent ligand-receptor binding assay was employed to map specificity of binding of Kinin and CAPA-1 within M. persicae and M. rosae. Flurophore-labelled kinin (kinin-F) and CAPA-1 (CAPA-1-F) revealed the neuropeptides to bind to the circular and longitudinal muscles of the aphid gut. Both the kinin-F and CAPA-1-F signals were displaced by excess unlabelled peptide in the ligand competition assay, thus confirming specificity of binding. Additional labelling with rhodamine phalloidin acted to confirm the gut muscle as the site of binding. Interestingly, specific kinin-F and CAPA-1-F binding of the gut musculature was not evident under low magnification (×10). The presence of smaller cells, running the length of the gut, were detected as a site of kinin-F binding, although were not a site of CAPA-1-F binding. (Data not shown.)

In addition, CAPA-1-F specific binding was detected in a region of the aphid midgut (stomach) closest to the foregut. Staining was abrogated when outcompeted with unlabelled 10⁻⁵M Capa (not shown).

Receptor mapping of the M. persicae brain and ventral nerve cord (VNC) revealed kinin-F staining apparent in a bilateral symmetrical ‘ladder’ of neuronal clusters (2-3 neurons) and a set of baso-lateral neurons in the suboesophageal ganglion. Staining was also apparent in symmetrical pairs of neurons/neuronal clusters in the ventro- to dorso-lateral protocerebrum. Little to no kinin-F staining was observed in the VNC with the exception of a set of cells in the most distal tip of the abdominal ganglion. In contrast, no specific staining with kinin-F was observed in the brain or VNC of M. rosae. Labelling with CAPA-1-F revealed no sites of receptor binding in either the brain or the VNC of both species (Data not shown).

Desiccation Stress

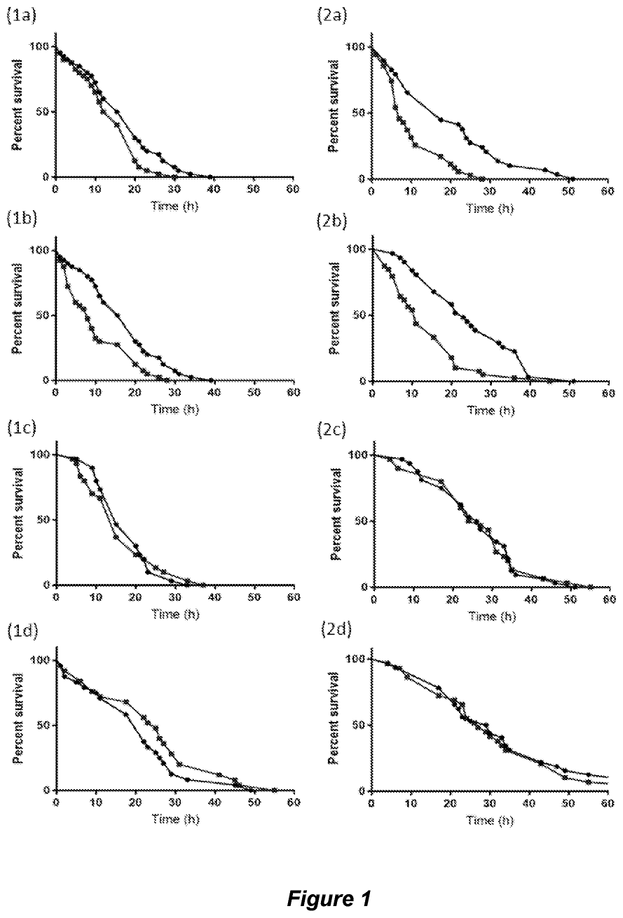

The CAP2b analogues 1895 and 2129 significantly increased desiccation/starvation mortality in both species (Table 2, FIG. 1). Here, treatment with 1895 acted to reduce the LTime₅₀ by 3.5 h and 9.6 h in M. persicae and M. rosae respectively, and median survival by 4.0 h and 10.5 h respectively (Table 2). Treatment with 2129 acted to reduce the LTime₅₀ by 7.1 h and 11.6 h in M. persicae and M. rosae respectively, and median survival by 9.8 h and 12.8 h respectively (Table 2). None of the kinin analogues significantly affected desiccation/starvation mortality in either species (Table 2).

Topical application of peptide 2125 under conditions of dessication/starvation conditions also resulted in significantly increased mortality for M. persicae (data not shown).

Cold Stress

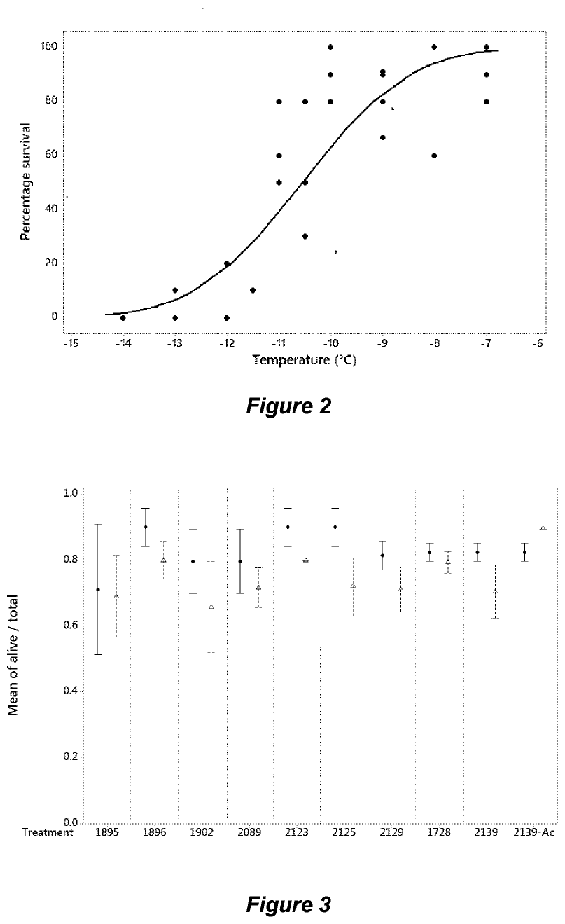

A survival curve was calculated for M. persicae (FIG. 2) and the LT₃₀ (discriminating temperature) calculated as −9.7° C. There was a significant effect of ‘Type’ (control vs. treatment) on the cold stress survival of M. persicae following cold shock at the discriminating temperature of −9.7° C. (GLM DF=1, χ²=5.9844, p=0.014), indicating that all analogues are efficient at increasing the mortality of test aphids under conditions of cold stress. However, there was no effect of the factor ‘Treatment’ (peptide analogue) on aphid cold stress survival (GLM DF=9, χ²=7.8355, p=0.551), indicating that all analogues appear equivalent in their effect, with no analogue having a stronger effect than another. Interestingly, treatment with analogue 2139-AC implies a reverse effect on aphid survival (FIG. 3), acting to increase survival relative to the control. However, further examination restricted to this case against its control proved non-significant (GLM DF=1, χ²=7.8355, p=0.3771). It must be concluded that all studied analogues increase the mortality of aphids under conditions of cold stress, with the exception of 2139-Ac, although no individual peptide is significantly more powerful in its effect.

Effect of Feeding with Peptide on Aphid Life Span and Fecundity

Aphids were reared on artificial diet, alone or supplemented with native CAPA neuropeptide, 1895 or 2129. A control group was reared on a host plant. Results are shown in Table 3.

Administration of the CAP2b analogues via an artificial diet acted to significantly reduce the lifetime fecundity of M. persicae, with analogue 1895 almost halving lifetime reproductive output when compared to control aphids reared on an artificial diet (6.4±2.0 nymphs compared to 11.7±1.5). Daily fecundity was also reduced from 1.5±0.1 nymphs when reared on an artificial diet to 1.1±0.1 and 1.2±0.1 nymphs when reared on an artificial diet containing analogues 1895 and 2129 respectively. Analogue treatment had little to no impact on life span, age at first reproduction and days as a reproducing adult.

Topical Application by Spraying

Results of the topical application by spraying (in the absence of additional external stress conditions) were as follows:

Application by Air Brush:

| Treatment | % Lethality |
|---|---|
| Airspray control | 0 |
| Vehicle control (Tween 24) | 0 |
| 1895 | 27 |
| 2129 | 30 |
| 1895 +2129 | 40 |
| Imadocloprid | 100 |

Application by Potter Spray Tower:

| Treatment | % Lethality |
|---|---|
| Vehicle control | 5.6 |
| (ATPlus UEP 100 LQ-(CQ)) | |
| 1895 | 34 |
| 2129 | 32 |
| 1895 + 2129 | 39 |
| Imadocloprid | 93 |

Topical Application of Peptides 2315 and 2320

Peptides were applied topically to insect abdomen by microinjector and the insects returned to the plant. Mortality was assessed after 24 hours. Separate controls were performed for each experiment. A group of 9 or 10 insects was used for each time point, and all experiments were performed 3 times.

Results are shown below:

| Peptide | Dead (24 h) |
|---|---|
| 2315 | 33.3% |
| Control | 20.7% |
| 2320 | 33.3% |
| Control | 6.7% |

Effect of Feeding with Peptides 2315, 2316, 2320 and 2129

Aphids were reared on artificial diet, alone or supplemented with peptide 2129, 2315, 2316 or 2320. A control group (no test peptide) was reared on a host plant. No other stress conditions were applied.

For peptides 2315, 2316 and 2320, mortality was assessed at 48, 72, 96, 120 and 144 hours. A group of 9 or 10 insects was used for each time point, and all experiments were performed at least 4 times.

Results are shown below:

| | Dead | Dead | Dead | Dead | Dead |
|---|---|---|---|---|---|
| Peptide | (48 h) | (72 h) | (96 h) | (120 h) | (144 h) |
| 2315 | 45.0% | 60.0% | 70.0% | 77.5% | 80.0% |
| 2316 | 86.0% | 88.0% | 90.0% | 92.0% | 92.0% |
| 2320 | 70% | 80% | 80% | 80% | 85% |
| None | 37.9% | 49.9% | 53.6% | 58.1% | 62.6% |

For peptide 2129, treatment resulted in a highly significant reduction in aphid longevity after the first 24 h, with ingestion of 2129 resulting in a percentage survival of 73.8% after 24 h and a change in mortality of 19.7% relative to the control.

### DISCUSSION

Neuropeptides are regulators of critical life processes in insects and, due to their high specificity, hold great potential in the drive for target-specific and environmentally friendly insecticidal agents.⁵ In pursuit of the development of aphid-specific neuropeptidomimetic-based insecticides, the current study mapped kinin and CAPA (CAPA-1) neuropeptide binding sites within M. persicae and M. rosae to determine neuropeptide function. CAP2b and kinin biostable analogues were subsequently assayed for target-insect-specificity and an ability to reduce aphid pest fitness, including in the presence and absence of a range of environmental stressors.

Receptor mapping employing fluorescently labelled kinin revealed the gut musculature as a main target for kinin activity in both M. persicae and M. rosae, as previously shown for M. persicae.⁷ Additional areas in the brain and VNC were also indicated in M. persicae. In the pea aphid, Acyrthosiphon pisum, it is thought that kinin regulates gut motility, digestive enzyme release, fluid cycling and nutrient transport across the gut.³⁴ Indeed, kinin analogues have shown great potential in the laboratory for their aphicidal properties, acting as antifeedant agents during artificial diet trials on the pea aphid (A. pisum).³⁴ Interestingly, whilst kinin analogues, including the present analogue 1728, have displayed prior antifeedant potential, none of the kinin analogues in the current study acted to reduce aphid fitness under desiccation and starvation stress conditions.

As with receptor mapping of kinin activity, receptor mapping with fluorescently labelled CAP2b revealed the muscles of the aphid midgut as the target for neuropeptide binding, with no CAP2b receptor binding detected in the aphid brain or VNC. However, in contrast to the kinin analogues, CAP2b analogues displayed greater promise in stress tolerance assays, with analogues 1895 (2Abf-Suc-FGPRLa) (SEQ ID NO: 13), 2129 (2Abf-Suc-ATPRIa) (SEQ ID NO: 12) and 2125 (2Abf-Suc-FT[Oic]RV-NH₂) (SEQ ID NO: 10) acting to expedite aphid (M. persicae and M. rosae) mortality under conditions of desiccation and/or starvation stress. Furthermore, all tested analogues (kinin and CAP2b), with the exception of 2139-Ac, enhanced M. persicae mortality under cold stress conditions, although were all considered equivalent in the strength of their effect.

Peptide 2315, 2316 and 2320 were found to increase mortality in the absence of additional stressors.

Neuropeptides of the CAPA family have roles in the stimulation of fluid secretion in Malpighian (renal) tubules⁴⁷ and, more recently, have been linked to desiccation and cold tolerance in Drosophila.¹⁷ Unlike most insects, aphids lack Malpighian tubules;⁴⁸ organs with vital roles in osmoregulation, detoxification and immunity.^{49,50} Due to this secondary loss of Malpighian tubules in the aphids, key osmoregulatory roles have been reassigned to other organs, particularly the aphid gut.⁵⁰ Receptor mapping assays performed in the current study offer support to this, highlighting the presence of CAPA receptors along the aphid gut and implicating the gut as a primary target for CAPA neuropeptide action. The role of CAPA neuropeptides in osmoregulation further offers explanation for the relative effectiveness of the CAP2b analogues tested in expediting aphid mortality under conditions of desiccation stress.

### REFERENCES

A number of publications are cited above in order to more fully describe and disclose the invention and the state of the art to which the invention pertains. Full citations for these references are provided below. The entirety of each of these references is incorporated herein.
Pimentel D, Acquay H, Biltonen M, Rice P, Silva M, Nelson J, Lipner V, Giordano S, Horowitz A and Damore M, Environmental and economic costs of pesticide use. Bioscience 42: 750-760 (1992).
Wilson C and Tisdell C, Why farmers continue to use pesticides despite environmental, health and sustainability costs. Ecol Econ 39: 449-462 (2001).
Altstein M and Nassel D R, Neuropeptide signaling in insects. Adv Exp Med Biol 692: 155-165 (2010).
Nachman R J and Pietrantonio P V, Interaction of mimetic analogs of insect kinin neuropeptides with arthropod receptors. Adv Exp Med Biol 692: 27-48 (2010).
Audsley N and Down R E, G protein coupled receptors as targets for next generation pesticides. Insect Biochem Molec 67: 27-37 (2015).
Holman G M, Nachman R J and Coast G M, Isolation, characterization and biological activity of a diuretic myokinin neuropeptide from the housefly, Musca domestica. Peptides 20:1-10 (1999).
Halberg K A, Terhzaz S, Cabrero P, Davies S A and Dow J A T, Tracing the evolutionary origins of insect renal function. Nat Commun 6 (2015).
Schoofs L, Vanden Broeck J and De Loof A, The myotropic peptides of Locusta migratoria: structures, distribution, functions and receptors. Insect Biochem Mol Biol 23: 859-881 (1993).
Coast G M, Holman G M and Nachman R J, The diuretic activity of a series of cephalomyotropic neuropeptides, the achetakinins, on isolated Malpighian tubules of the house cricket Acheta domesticus. J Insect Physiol 36: 481-488 (1990).
Dow J A, Insights into the Malpighian tubule from functional genomics. J Exp Biol 212: 435-445 (2009).
Harshini S, Nachman R J and Sreekumar S, Inhibition of digestive enzyme release by neuropeptides in larvae of Opisina arenosella (Lepidoptera: Cryptophasidae). Comp Biochem Physiol B132: 353-358 (2002).
Nachman R J, Coast G M, Douat C, Fehrentz J A, Kaczmarek K, Zabrocki J, Pryor N W and Martinez J, A C-terminal aldehyde insect kinin analog enhances inhibition of weight gain and induces significant mortality in Helicoverpa zea larvae. Peptides 24: 1615-1621 (2003).
Cannell E, Dornan A J, Halberg K A, Terhzaza S, Dow J A T and Davies S A, The corticotropin-releasing factor-like diuretic hormone 44 (DH44) and kinin neuropeptides modulate desiccation and starvation tolerance in Drosophila melanogaster. Peptides 80: 96-107 (2016).
Zandawala M, Marley R, Davies S A and Nassel D R, Characterization of a set of abdominal neuroendocrine cells that regulate stress physiology using colocalized diuretic peptides in Drosophila. Cell Mol Life Sci 75: 1099-1115 (2018).
Huesmann G R, Cheung C C, Loi P K, Lee T D, Swiderek K M and Tublitz N J, Amino acid sequence of CAP2b, an insect cardioacceleratory peptide from the tobacco hawkmoth Manduca sexta. FEBS Lett 371: 311-314 (1995).
Davies S A, Cabrero P, Povsic M, Johnston N R, Terhzaz S and Dow J A T, Signaling by drosophila capa neuropeptides. Gen Comp Endocr 188: 60-66 (2013).
Terhzaz S, Teets N M, Cabrero P, Henderson L, Ritchie M G, Nachman R J, Dow J A T, Denlinger D L and Davies S A, Insect capa neuropeptides impact desiccation and cold tolerance. Proc Natl Acad Sci 201501518 (2015).
Terhzaz S, Alford L, Yeoh J G C, Marley R, Dornan A T, Dow J A T and Davies S A, Renal neuroendocrine control of desiccation and cold tolerance by Drosophila suzukii. Pest Manag Sci 74: 800-810 (2017).
Predel R, Wegener C, Biology of the CAPA peptides in insects. Cell Mol Life Sci 63: 2477-2490 (2006).
Predel R, Nachman R J, The FXPRLamide (Pyrokinin/PBAN) Peptide Family, in Handbook of biologically active peptides, ed. by Kastin, A J, Elsevier; New York, pp. 207-212 (2006).
Jiang H, Wei Z, Nachman R J, Adams M E and Park Y, Functional phylogenetics reveals contributions of pleiotropic peptide action to ligand-receptor coevolution. Sci Rep 4: 6800 (2014).
Jiang H, Wei Z, Nachman R J, Kaczmarek K, Zabrocki J and Park Y, Functional characterization of five different PRXamide receptors of the red flour beetle Tribolium castaneum with peptidomimetics and identification of agonists and antagonists. Peptides 68: 246-252 (2015).
Zhang Q, Nachman R J, Kaczmarek K, Zabrocki J and Denlinger D L, Disruption of insect diapause using agonists and an antagonist of diapause hormone. Proc Natl Acad Sci USA 108: 16922-16926 (2011)
Cornell M J, Williams T A, Lamango N S, Coates D, Corvol P, Soubier F, Hoheisel J, Lehrach H and Isaac R E, Cloning and expression of an evolutionary conserved single-domain angiotensin converting enzyme from Drosophila melanogaster. J Biol Chem 270: 13613-13619 (1995).
Lamango N S, Nachman R J, Hayes T K, Strey A and Isaac R E, Hydrolysis of insect neuropeptides by an angiotensin converting enzyme from the housefly, M. domestics. Peptides 18: 47-52 (1997).
Nachman R J, Strey A, Isaac E, Pryor N, Lopez J D, Deng J G and Coast G M, Enhanced in vivo activity of peptidase-resistant analogs of the insect kinin neuropeptide family. Peptides 23: 735-745 (2002).
Nachman R J, Isaac R E, Coast G M, Roberts V A, Lange A, Orchard I, Holman G M and Favrel P, Active conformation and mimetic analog development for the Pyrokinin/PBAN and Myosuppressin insect neuropeptide families. In: Hedin P A, Hollingworth R M, Masler E P, Miyamoto J, Thompson D G (eds) Phytochemicals for pest control, ACS Symposium Series 658, ACS, Washington, D.C. pp 277-291 (1997).
Nachman R J, Isaac R E, Coast G M and Holman G M, Aib-containing analogues of the insect kinin neuropeptide family demonstrate resistance to an insect angiotensin-converting enzyme and potent diuretic activity. Peptides 18: 53-57 (1997).
Taneja-Bageshwar S, Strey A, Zubrzak P, Pietrantonio P V and Nachman R J, Comparative structure—activity analysis of insect kinin core analogs on recombinant kinin receptors from Southern cattle tick Boophilus microplus (Acari: Ixodidae) and mosquito Aedes aegypti (Diptera: Culicidae). Arch Insect Biochem Physiol 62: 128-140 (2006).
Taneja-Bageshwar S, Strey A, Isaac E R, Coat G M, Zubrzak P, Pietrantonio P V, Nachman R J, Biostable agonists that match or exceed activity of native insect kinins on recombinant arthropod GPCRs. Gen Comp Endocr 162: 122-128 (2009).
Holmes S P, He H, Chen A C, Ivie G W and Pietrantonio P V, Cloning and transcriptional expression of a leucokinin-like peptide receptor from the southern cattle tick Boophilus microplus (Acari: Ixodidae). Insect Mol Biol 9: 457-465 (2000).
Holmes S P, Barhoumi R, Nachman R J and Pietrantonio P V, Functional analysis of a G protein-coupled receptor from the southern cattle tick Boophilus microplus (Acari: Ixodidae) identifies it as the first arthropod myokinin receptor. Insect Mol Biol 12: 27-38 (2003).
Pietrantonio P V, Jagge C, Taneja-Bageshwar S, Nachman R J and Barhoumi R, The mosquito Aedes aegypti (L.) leucokinin receptor is a multiligand receptor for the three Aedes kinins. Insect Mol Biol 14: 55-67 (2005).
Smagghe G, Mandian K, Zubrzak P and Nachman R J, Antifeedant activity and high mortality in the pea aphid Acyrthosiphon pisum (Hemiptera: Aphidae) induced by biostable insect kinin analog. Peptides 31: 498-505 (2010).
Zhang C, Qu Y, Wu X, Song D, Ling Y and Yang X, Design, synthesis and aphicidal activity of N-terminal modified insect kinin analogs. Peptides 68: 233-238 (2015).
Radford J C, Davies S A and Dow J A, Systematic G-protein-coupled receptor analysis in Drosophila melanogaster identifies a leucokinin receptor with novel roles. J Biol Chem 277: 38810-38817 (2002).
Terhzaz S, Cabrero P, Robben J H, Radford J C, Hudson B D, Milligan G, Dow J A and Davies S A, Mechanism and function of Drosophila capa GPCR: a desiccation stress-responsive receptor with functional homology to human neuromedinU receptor. PLoS One 7(1): e29897 (2012).
Christie A E, In silico analyses of peptide paracrines/hormones in Aphidoidea. Gen Comp Endocr 159: 67-79 (2008).
Nault L R, Arthropod transmission of plant viruses: A new synthesis. Ann Entomol Soc Am, 90: 521-541 (1997).
van Emden H F and Harrington R, Aphids as Crop Pests, CABI Publishing, Wallingford, 717 p (2007).
Blackman R L and Eastop V F, Aphids on the World's Crops: An Identification Guide, John Wiley & Sons Ltd, Chichester, UK. (2000).
Dow J A T, Maddrell S H P, Gortz A, Skaer N J V, Brogan S and Kaiser K, The Malpighian tubules of Drosophila melanogaster—a novel phenotype for studies of fluid secretion and its control. J Exp Biol 197: 421-428 (1994).
Clough M S, Bale J S and Harrington R, Differential cold hardiness in adults and nymphs of the peach-potato aphid Myzus-Persicae. Ann Appl Biol 116: 1-9 (1990).
Powell S J and Bale J S, Intergenerational acclimation in aphid overwintering. Ecol Entomol 33: 95-100 (2008).
Sinclair B J and Chown S L, Rapid cold-hardening in a Karoo beetle, Afrinus sp. Physiol Entomol 31: 98-101 (2006).
Terblanche J S, Clusella-Trullas S, Deere J A and Chown S L, Thermal tolerance in a south-east African population of the tsetse fly Glossina pallidipes (Diptera, Glossinidae): Implications for forecasting climate change impacts. J Insect Physiol 54: 114-127 (2008).
Davies S A, Huesmann G R, Maddrell S H, O'Donnell M J, Skaer N J, Dow J A T and Tublitz N J, CAP2b, a cardioacceleratory peptide, is present in Drosophila and stimulates tubule fluid secretion via cGMP. Am J Physiol 269: R1321-R1326 (1995).
Douglas A E, Nutritional physiology of aphids. Adv Insect Physiol 31: 73-140 (2003).
Beyenbach K W, Skaer H and Dow J A, The developmental, molecular, and transport biology of Malpighian tubules. Annu Rev Entomol 55: 351-374 (2010).
Jing X, White T A, Yang X and Douglas A E, The molecular correlates of organ loss: The case of insect Malpighian tubules. Biol Letters 11: 20150154 (2015).
Sadeghi A., Van Damme, E. J. M. and Smagghe G. (2009) Evaluation of the susceptibility of the pea aphid, Acyrthosiphon pisum, to a selection of novel biorational insecticides using an artificial diet. Journal of Insect Science 9:65.
Van Emden F (2009). Artificial diet for aphids—thirty years' experience. REDIA, XCII: 163-167
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

TABLE 1

| Code | Structure |
|---|---|
| CAP2b/PK | |
| 1895 | 2Abf-Suc-FGPRL-NH₂ |
| | (SEQ ID NO: 13) |
| 1896 | 2Abf-Suc-FTPRI-NH₂ |
| | (SEQ ID NO: 6) |
| 1902 | 2Abf-Suc-FKPRL-NH₂ |
| | (SEQ ID NO: 7) |
| 2089 | 2Abf-Suc-FTPRV-NH₂ |
| | (SEQ ID NO: 8) |
| 2123 | 2Abf-Suc-FT[Hyp]RV-NH₂ |
| | (SEQ ID NO: 9) |
| 2125 | 2Abf-Suc-FT[Oic]RV-NH₂ |
| | (SEQ ID NO: 10) |
| 2129 | 2Abf-Suc-ATPRI-NH₂ |
| | (SEQ ID NO: 12) |
| 2315 | ASG[βhL]VAFPRV-NH₂ |
| | (SEQ ID NO: 18) |
| 2316 | ASGL[βhL]AFPRV-NH₂ |
| | (SEQ ID NO: 19) |
| 2320 | ASG[βhL]V[βA]FPRV-NH₂ |
| | (SEQ ID NO: 20) |
| Kinin | |
| 1728 | [Aib]FF[Aib]WG-NH₂ |
| | (SEQ ID NO: 24) |
| 2139 | FF[Aib]WG-NH₂ |
| | (SEQ ID NO: 25) |
| 2139-AC | Acetyl-FF[Aib]WG-NH₂ |
| | (SEQ ID NO: 25) |

TABLE 3

| | | Age at 1st | | | | |
|---|---|---|---|---|---|---|
| | Life span | reproduction | Days as | Lifetime | Ave. daily | |
| | (d) | (d) | reproducing adult | fecundity | fecundity | n |
| Plant | 37.5 ± 1.7 | 8.8 ± 0.1 | 17.9 ± 0.9 | 68.8 ± 3.9 | 4.0 ± 0.2 | 40 |
| Artificial diet | 19.9 ± 0.9 | 11.0 ± 0.2 | 7.7 ± 0.7 | 11.7 ± 1.5 | 1.5 ± 0.1 | 12 |
| Native Kinin | 18.8 ± 1.3 | 11.1 ± 0.5 | 5.9 ± 1.0 | 9.7 ± 1.9 | 1.6 ± 0.2 | 9 |
| Native Capa | 21.3 ± 0.7 | 11.2 ± 0.2 | 7.6 ± 0.6 | 12.0 ± 1.2 | 1.6 ± 0.1 | 12 |
| 1895 | 19.8 ± 2.5 | 11.5 ± 0.5 | 5.8 ± 1.8 | 6.4 ± 2.0 | 1.1 ± 0.1 | 5 |
| 2129 | 21.0 ± 2.3 | 12.4 ± 0.7 | 7.17 ± 1.5 | 8.8 ± 2.0 | 1.2 ± 0.1 | 6 |

TABLE 2

| | Myzus persicae | LTime₅₀ (h) | Median ± IQR survival (h) | | |
|---|---|---|---|---|---|
| | Documented effect | control \| test | control \| test | X² | p |
| 1728 | No effect | 18.3 \| 25.0 | 22.0 ± 16.5 \| 26.0 ± 23.5 | 2.260 | 0.133 |
| 2139 | No effect | 18.3 \| 22.4 | 22.0 ± 16.5 \| 25.0 ± 21.0 | 1.341 | 0.247 |
| 2139-AC | No effect | 19.4 \| 16.7 | 22.5 ± 19.8 \| 20.0 ± 14.0 | 2.498 | 0.114 |
| 1895 | Increases | 15.9 \| 12.4 | 17.8 ± 12.0 \| 13.8 ± 11.8 | 3.948 | 0.047 |
| | mortality | | | | |
| 1896 | No effect | 22.2 \| 24.8 | 24.0 ± 0 9.0 \| 25.0 ± 10.5 | 1.939 | 0.164 |
| 1902 | No effect | 22.2 \| 21.8 | 24.0 ± 0 9.0 \| 23.5 ± 7.0 | 0.030 | 0.863 |
| 2089 | No effect | 10.9 \| 13.0 | 11.0 ± 12.0 \| 16.5 ± 10.3 | 2.197 | 0.138 |
| 2123 | No effect | 24.3 \| 21.1 | 25.0 ± 13.5 \| 21.0 ± 13.5 | 2.092 | 0.148 |
| 2125 | No effect | 10.8 \| 13.0 | 11.0 ± 12.0 \| 11.0 ± 12.0 | 1.309 | 0.253 |
| 2129 | Increases | 15.9 \| 8.8 | 17.8 ± 12.0 \| 8.0 ± 17.0 | 10.200 | 0.001 |
| | mortality | | | | |
| | Macrosiphum rosae | LTime₅₀ (h) | Median IQR survival (h) | | |
| | Documented effect | control \| test | control \| test | X² | p |
| 1728 | No effect | 31.7 \| 31.6 | 29.5 ± 22.0 \| 24.0 ± 28.5 | 0.431 | 0.512 |
| 2139 | No effect | 31.7 \| 29.8 | 29.5 ± 22.0 \| 27.0 ± 26.0 | 0.176 | 0.675 |
| 2139-AC | No effect | 44.0 \| 40.1 | 39.0 ± 37.0 \| 35.0 ± 32.0 | 0.210 | 0.647 |
| 1895 | Increases | 18.7 \| 9.1 | 17.5 ± 19.5 \| 7.0 ± 12.5 | 14.060 | <0.0001 |
| | mortality | | | | |
| 1896 | No effect | 28.9 \| 24.7 | 27.0 ± 17.3 \| 23.0 ± 18.5 | 0.065 | 0.799 |
| 1902 | No effect | 28.9 \| 26.4 | 27.0 ± 17.3 \| 25.0 ± 10.0 | 0.224 | 0.636 |
| 2089 | No effect | 23.2 \| 16.4 | 23.0 ± 17.0 \| 17.0 ± 21.3 | 2.002 | 0.157 |
| 2123 | No effect | 21.6 \| 19.1 | 24.0 ± 11.0 \| 16.0 ± 18.0 | 1.806 | 0.179 |
| 2125 | No effect | 21.6 \| 20.1 | 24.0 ± 9.25 \| 20.5 ± 18.0 | 0.215 | 0.643 |
| 2129 | Increases | 24.3 \| 12.7 | 23.8 ± 20.5 \| 11.0 ± 13.0 | 14.320 | >0.0001 |
| | mortality | | | | |

1. Use, as an insect control agent against hemipteran insects, of a compound having the formula:
R¹-L¹-Z^{a}—Z—R²
wherein:
Zₐ is a peptide of 1 to 8 amino acids, or is absent;
Z is a peptide having a sequence selected from:
A-Xa-PR-Xb;
F-Xc-PRL;
where Xa and Xc are independently G or T and Xb is I or V;
FTPRI;
FKPRL;
FTPRV;
FT[Hyp]RV; and
FT[Oic]RV;
L¹ is absent or is selected from C₁₋₆-alkylene, C₁₋₆-alkenylene and (poly)alkyleneglycol where each L¹ if present may be optionally substituted with one or more groups selected from oxo (═O), halogen, ═N and ═S;
R¹ is hydrogen, C₁₋₄ alkyl (e.g. methyl, ethyl, propyl, butyl), acetyl, formyl, benzoyl or trifluoroacetyl, —NHC₆₋₁₆-Aryl, or —NH—C₁₋₆-alkyl-C₆₋₁₀aryl, each of which may optionally be substituted with one or more groups selected from halogen, C₁₋₆-alkyl, or C₁₋₆-haloalkyl;
and R² is NH₂ or OR²a wherein R^{2a} is C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkyl-C₆₋₁₆-aryl, or C₁₋₆-haloalkyl, each of which may optionally be substituted with one or more groups selected from halogen, C₁₋₆-alkyl, or C₁₋₆-haloalkyl.

2. Use according to claim 1 wherein Z has the formula ATPR-Xb, where Xb is I or V.

3. Use according to claim 1 or claim 2 wherein Z has the formula ATPRI.

4. Use according to claim 1 wherein Z is FGPRL.

5. Use according to any one of claims 1 to 4 wherein Zₐ is absent.

6. Use according to any one of claims 1 to 5 wherein L¹ is C₁₋₆-alkylene optionally substituted with one or more groups selected from oxo (═O), halogen, ═N and ═S.

7. Use according to claim 6 wherein L¹ is —(C═O)C₁₋₄-alkylene-(C═O)—, e.g. L¹ is

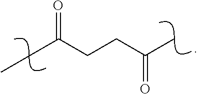

8. Use according to any one of claims 1 to 7 wherein R¹ is —NHC₁₋₁₈-alkyl, —NHC₆₋₁₆-Aryl, or —NH—C₁₋₆-alkyl-C₆₋₁₆aryl optionally substituted with one or more groups selected from halogen, C₁₋₆-alkyl, or C₁₋₆-haloalkyl.

9. Use according to claim 8 wherein R¹ is —NHC₆₋₁₆-Aryl optionally substituted with one or more groups selected from halogen, C₁₋₆-alkyl, or C₁₋₆-haloalkyl.

10. Use according to any one of claims 1 to 9 wherein R¹ is selected from N(H)npropyl, —N(H)ipropyl, —N(H)nbutyl, —N(H)isoamyl, —N(H)nhexyl, —N(H)noctyl, —N(H)t-octyl, —N(H)ndecyl, —N(H)ndodecyl, or N(H)-fluorenyl optionally substituted with one or more halogen groups.

11. Use according to any one of claims 1 to 10 wherein R¹ is:

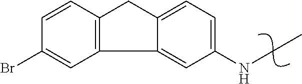 [SMILES: CCNc1ccc2c(c1)-c1cc(Br)ccc1C2]

12. Use according to any one of claims 1 to 11 wherein L¹-R¹ is 2Abf-Suc:

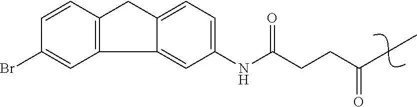 [SMILES: CCC(=O)CCC(=O)Nc1ccc2c(c1)-c1cc(Br)ccc1C2]

13. Use according to any one of claims 1 to 12 wherein R² is NH₂

14. Use according to claim 1 wherein the compound is:
2Abf-Suc-ATPRI-NH₂;
2Abf-Suc-FGPRL-NH₂;
2Abf-Suc-FTPRI-NH₂;
2Abf-Suc-FKPRL-NH₂;
2Abf-Suc-FTPRV-NH₂;
2Abf-Suc-FT[Hyp]RV-NH₂; or
2Abf-Suc-FT[Oic]RV-NH₂.

15. Use according to any one of claims 1 to 14 wherein said use is as an insecticide against hempiteran insects.

16. A method of increasing hemipteran insect mortality comprising contacting a hemipteran insect or hemipteran insect population with a compound as described in any one of claims 1 to 14.

17. A method of reducing cold tolerance, reducing desiccation stress tolerance, reducing starvation stress tolerance, and/or reducing fecundity of a hemipteran insect, or of a hemipteran insect population, comprising contacting a hemipteran insect or insect population with a compound as described in any one of claims 1 to 14.

18. Use of a compound as described in any one of claims 1 to 14 as a plant protection agent, for protecting a plant against hemipteran insects.

19. A method of inhibiting infestation of a plant by hemipteran insects comprising contacting the plant with a compound as described in any one of claims 1 to 14.

20. A method according to claim 19 wherein the compound is applied to the plant while the plant is free or substantially free of hemipteran insects.

21. A method of reducing hempiteran insect infestation of a plant, or of reducing hemipteran insect load on a plant, the method comprising contacting the plant with a compound as described in any one of claims 1 to 14.

22. A compound having the formula:
R¹-L¹-Z^{a}—Z—R²
wherein:
Zₐ is a peptide of 1 to 12 amino acids, or is absent;
Z is a peptide having a sequence selected from:
A-Xa-PR-Xb, where Xa is G or T and Xb is I or V;
FTPRV;
FT[Hyp]RV; and
FT[Oic]RV.
L¹ is absent or is selected from C₁₋₆-alkylene, C₁₋₆-alkenylene and (poly)alkyleneglycol where each L¹ if present may be optionally substituted with one or more groups selected from oxo (═O), halogen, ═N and ═S.
R¹ is hydrogen, C₁₋₄ alkyl (e.g. methyl, ethyl, propyl, butyl), acetyl, formyl, benzoyl or trifluoroacetyl, —NHC₁₋₁₈-alkyl, —NHC₆₋₁₆-Aryl, or —NH—C₁₋₆-alkyl-C₆₋₁₀aryl, each of which may optionally be substituted with one or more groups selected from halogen, C₁₋₆-alkyl, or C₁₋₆-haloalkyl.
and R² is NH₂ or OR²a wherein R^{e}a is C₁₋₆-alkyl (e.g. methyl, ethyl, propyl, butyl, pentyl or hexyl), C₃₋₆-alkenyl, C₆₋₁₆-aryl, C₁₋₆-alkyl-C₆₋₁₆-aryl, or C₁₋₆-haloalkyl, each of which may optionally be substituted with one or more groups selected from halogen, C₁₋₆-alkyl, or C₁₋₆-haloalkyl.

23. A compound according to claim 22 wherein Z has the formula ATPR-Xb, where Xb is I or V.

24. A compound according to claim 23 wherein Z has the formula ATPRI.

25. A compound according to claim 22 wherein Z is FGPRL.

26. A compound according to any one of claims 22 to 25 wherein Zₐ is absent.

27. A compound according to any one of claims 22 to 26 wherein L¹ is C₁₋₆-alkylene optionally substituted with one or more groups selected from oxo (═O), halogen, ═N and ═S.

28. A compound according to any one of claims 22 to 27 wherein L¹ is —(C═O)C₁₋₄-alkylene-(C═O)—, e.g. L¹ is

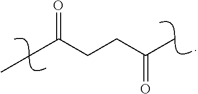

29. A compound according to any one of claims 22 to 28 wherein R¹ is —NHC₁₋₁₈-alkyl, —NHC₆₋₁₆-Aryl, or —NH—C₁₋₆-alkyl-C₆₋₁₆-aryl optionally substituted with one or more groups selected from halogen, C₁₋₆-alkyl, or C₁₋₆-haloalkyl.

30. A compound according to any one of claims 22 to 29 wherein R¹ is —NHC₆₋₁₆-Aryl optionally substituted with one or more groups selected from halogen, C₁₋₆-alkyl, or C₁₋₆-haloalkyl.

31. A compound according to any one of claims 22 to 30 wherein R¹ is selected from N(H)npropyl, —N(H)ipropyl, —N(H)nbutyl, —N(H)isoamyl, —N(H)nhexyl, —N(H)noctyl, —N(H)t-octyl, —N(H)ndecyl, —N(H)ndodecyl, or N(H)-fluorenyl optionally substituted with one or more halogen groups.

32. A compound according to any one of claims 22 to 31 wherein R¹ is:

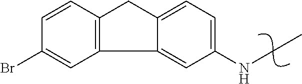 [SMILES: CCNc1ccc2c(c1)-c1cc(Br)ccc1C2]

33. A compound according to any one of claims 22 to 32 wherein L¹-R¹ is 2Abf-Suc:

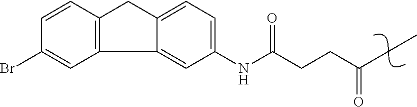 [SMILES: CCC(=O)CCC(=O)Nc1ccc2c(c1)-c1cc(Br)ccc1C2]

34. A compound according to any one of claims 22 to 33 wherein R² is NH₂

35. A compound according to claim 22 which is:
2Abf-Suc-ATPRI-NH₂;
2Abf-Suc-FTPRV-NH₂;
2Abf-Suc-FT[Hyp]RV-NH₂; or
2Abf-Suc-FT[Oic]RV-NH₂.

36. A compound having the formula:
R¹-L₁-Z^{a}—Z—R²
wherein:
Z^{a} is a peptide of 1 to 12 amino acids, or is absent;
Z is a peptide having the formula:
ASG-X4-X5-X6-FPRV
wherein:
X4 is L, [βhL], [βhA] or [βhF];
X5 is V, [βhL], [βhV], [βhA] or [βhF];
X6 is A or [βA].
L¹ is absent or is selected from C₁₋₆-alkylene, C₁₋₆-alkenylene and (poly)alkyleneglycol where each L¹ if present may be optionally substituted with one or more groups selected from oxo (═O), halogen, ═N and ═S.
R¹ is hydrogen (which may be designated “H” or “Hy”), C₁₋₄ alkyl (e.g. methyl, ethyl, propyl, butyl), acetyl, formyl, benzoyl or trifluoroacetyl, —NHC₁₋₁₈-alkyl, —NHC₆₋₁₆-Aryl, or —NH—C₁₋₆-alkyl-C₆₋₁₀aryl, each of which may optionally be substituted with one or more groups selected from halogen, C₁₋₆-alkyl, or C₁₋₆-haloalkyl.
and R² is NH₂ or OR²a wherein R^{e}a is C₁₋₆-alkyl (e.g. methyl, ethyl, propyl, butyl, pentyl or hexyl), C₃₋₆-alkenyl, C₆₋₁₆ aryl, C₁₋₆-alkyl-C₆₋₁₆-aryl, or C₁₋₆-haloalkyl, each of which may optionally be substituted with one or more groups selected from halogen, C₁₋₆-alkyl, or C₁₋₆-haloalkyl.

37. A compound according to claim 36 wherein the peptide Z has the formula:
ASG-X4-VAFPRV, wherein X4 is L, [βhL], [βhA] or [βhF];
ASGL-X5-AFPRV, wherein X5 is V, [βhL], [βhV], [βhA] or [βhF]; or
ASG-X4-V-X6-FPRV wherein X4 is L, [βhL], [βhA] or [βhF]; and X6 is A or [βA].

38. A compound according to claim 36 or claim 37 wherein:
X4 is L or [βhL];
X5 is V or [βhL];
X6 is A or [βA].

39. A compound according to any one of claims 36 to 38 wherein the peptide Z has the sequence:
ASG[βhL]VAFPRV;
ASGL[βhL]AFPRV; or
ASG[βhL]V[βA]FPRV.

40. A compound according to any one of claims 36 to 39 wherein Z^{a} is absent.

41. A compound according to any one of claims 36 to 40 wherein L¹ is absent.

42. A compound according to any one of claims 36 to 41 wherein R¹ is hydrogen or acetyl.

43. A compound according to any one of claims 36 to 42 wherein R² is NH₂

44. A compound according to claim 36 which is:
Hy-ASG[βhL]VAFPRV-NH₂ [2315];
Hy-ASGL[βhL]AFPRV-NH₂ [2316]; or
Hy-ASG[βhL]V[βA]FPRV-NH₂ [2320].

45. Use, as an insect control agent, of a compound according to any one of claims 36 to 44.

46. Use according to claim 45 wherein said use is as an insecticide against hempiteran insects.

47. A method of increasing hemipteran insect mortality comprising contacting a hemipteran insect or hemipteran insect population with a compound as described in any one of claims 36 to 44.

48. A method of reducing cold tolerance, reducing desiccation stress tolerance, reducing starvation stress tolerance, and/or reducing fecundity of a hemipteran insect, or of a hemipteran insect population, comprising contacting a hemipteran insect or insect population with a compound as described in any one of claims 36 to 44.

49. Use of a compound as described in any one of claims 36 to 44 as a plant protection agent, for protecting a plant against hemipteran insects.

50. A method of inhibiting infestation of a plant by hemipteran insects comprising contacting the plant with a compound as described in any one of claims 36 to 44.

51. A method according to claim 50 wherein the compound is applied to the plant while the plant is free or substantially free of hemipteran insects.

52. A method of reducing hempiteran insect infestation of a plant, or of reducing hemipteran insect load on a plant, the method comprising contacting the plant with a compound as described in any one of claims 36 to 44.

53. A composition, e.g. an insect control composition or plant protection composition, comprising a compound according to any one of claims 22 to 25 or 36 to 44 in admixture with one or more solvents, carriers, diluents, adjuvants, preservatives, dispersants, emulsifying agents, or synergists.

54. A composition according to claim 53 which is an aqueous composition.
